SEQUENCE LISTING (37 sequences)

SEQ ID NO: 1 - DNA, 20 bp, Zea mays
  misc_feature (1)..(20): 5′ genome-insert junction
gtcgtggaggaaacactgat

SEQ ID NO: 2 - DNA, 20 bp, Zea mays
  misc_feature (1)..(20): 3′ insert-genome junction
gttattaagtcggggagggg

SEQ ID NO: 3 - DNA, 60 bp, Zea mays
  misc_feature (1)..(60): longer 5′ genome-insert sequence
ggaggggagcgtgagttggagtcgtggaggaaacactgatagtttaaactgaaggcggga

SEQ ID NO: 4 - DNA, 60 bp, Zea mays
  misc_feature (1)..(60): longer 3′ insert-genome junction
catggccgtatccgcaatgtgttattaagtcggggaggggagcgtgagttgtagtcgcgg

SEQ ID NO: 5 - DNA, 1024 bp, Zea mays
  misc_feature (1)..(1024): 5′ genomic flanking sequence
cttgaaaggatgaaccatgaaaatttagttgcgatgtttccatgtagtgaaccgagatgtcttatcagtaagtaaataggttaacatcaagaaagcacatttaataaaaaattggaaaggggacatacattaattaactatctaaaagtagtacacagaattagacatttagacataatatggtttgctaaatacgtagtaatctatataaacaaaattaaagttgtgtatttctaagacatgcatacgtacacaaacagaacatattgaaactagcctttgttgaacaatttttaacagcaaatgtggttttggtttgttaatattgcaagttattatgagtattcaacctagtaaattgaataatccttgcctgatatattgaatctatttgtatatatctagctagatacaattattagaacatgcttttattgtctacatatgtacccatgtaacatgaatagaaaataaataggtatgcttatcgtagagtatctatgaccatgtagcaagaaaaataagttaatatagagataaatcaatgggcaaaaaataaaatttgggggaaagaatctcactgctactactcttccactacaaagaacaacaaggttggttccaattccacttccggaacaatcagcgcaattgctccatccatccacagctgccaccgagactaggtgacacgatccaagccgccatggcgtcggctaagcctctacgctctagactgtgctggtggcactagggagactacgaaggttcacctgcaccttgaaggactagttgaggaaggtgttggcgaggatgttgaggggtttggacgatgaagcaccgtctagggaacaattgttctggcccgtgacgaggtgctgcaacgccatctccgccgccggagagacgtgcacgctctctgacgtcacatgtgagaggctatggttgtagcttcaatcgttggcggtgtgggctggtccggctcgatgtgaggggaggggagcgtgagttggagtcgtggagg

SEQ ID NO: 6 - DNA, 1037 bp, Zea mays
  misc_feature (1)..(1037): 3′ genomic flanking sequence
cggggaggggagcgtgagttgtagtcgcggtgtgatcttgacccgacgcatgatttaacaggaggtgactcgtgaaccttggacgacgatcgtgtggctgggaaaccaagggatgatatggacgcctcgagtgacgagatttgctcgtagctctattgatagcaggggcgaagctacgacttgttgatggtgtcaaatggcaccgataagttttagaaaatacaatgtaaaatattgtttacaatgttgatggcattcctataaaactgtgctgacactagcaaaacttaaggctggcttcgccccttattgatagtaaggctatggcctggagatcatgtatataatcgtttaaaatattattttatattatagactaagttatgtatatagtaaaataaaatttaaaatagaagatggaacgagcaaggagataacctaatgtgctgagacatgagagcgttatgccggaccgggccagaatcttcgcgcctccccggtccacagaaaccgcggtccgccagagcgcggcgcgcccagcgtccctgctcccaaagtcccatgcggcatgcgcccggcccaattggaaaataaagcaccgccgcatatcccgccctttccttttgtcggatggcgcgtcacggcgtttttccccataaaaaattcagaaatatataaacaccagacatgcgcccgcgcgctcttccctccctgccgcccgcgtcctcctctccacaccaatgcggccaccaccgcacgtcgccgcctccacctcgcctgccctccacgccgggtcgtcccggcgccggcctcaccaccgcggccggacgatgatgtccgtctcctgctgccacttggcggcgctcgtgtccatcctcgcggcgggcgcggctacggcgttgctcaccttgtccctaccctcttctcctggcgcgtccacgacgaggagaaccgacttcgttggagcattgtcagtggcaaacgagaccccgccaccgccgcacttgtcagcgcctccggcacccgccacgccgccc

SEQ ID NO: 7 - DNA, 10435 bp, Artificial Sequence
  Heterologous inserted DNA from plasmid 15777
aaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggaccgctaggacgatggtgtgatgtgggaacacgaagaaaacatgaggaaaaaatattaaaatgaatttcccacttaaaatgcatcaaataaaaaaaataaagaaacgaccgggaatagacacagggtttgtgaactagctagggcaaacatcatatggtcccttgctgatgcacaagtacattgagatgtcatttcaattctgtgcatcatatgcatgtggtcccttgctgaatattactcttgaaatatctaccagtgccaatctattgcatgacttaattaattcacaggttttgttgattacattattagtaagcttgagagcacaagctcaatggatttttctataaatggggatcattttgcaattttctttgtcgtgcaaagttagccttctttattactacttctgtttttaaatatacgatcctattgacttttggtcatatatttaaccatgtatcttatttagatagtttgcgcaaatatatataccttcaatgataaaattagttacaatgaaacaaatgatatttacgcaattctttttactaaacaagtcacaagaagtacctgcagcaatatatgttggaaccgtgcagtagatcgagcctagctacgcaaaaaaacaaaaagagaaaaaaagggaaaggaaaaacattaatcatgcatgagcagtatgcccggcaactggaatttgtcaaagatatggggagaggagaataatacaagtactactactacctagctctaccatgcatatgcacccaaaggcaaactggattattggataaagcacagatgctggcaaaacaatccttaagcctcccctccctgcttctttatttttgggcagcctctaccggacggtgccgtggtccattggaccagtaggtggcgacatacatggtttgggttaagtctaggagagcagtgtgtgtgcgcgcgcaagagagagagactgtgagtctgggagtagccctctcccctcctttggccatcttcctcgtgtatatgcatatatgcatcatcgcaacggtgtatatttgtggtgtggcgggtgtggcattggattgcccccattttggctcgtgcttcccagttagggtaaaacctgtggtaaacttgctagccccacgccaaagttacccttctttattgttgaaagggagaggaggtgtgtgaattgtgatggagggagagagagagagatagaaagagagatgtgtgtcaaagcaagcaagaaaccagtttcacaaagagctactactagtactagtgtactactgtggtacagtgcccaatgtcctttctccggactcgactccactaatattctcctcttctcgcgcggctcgttatattctcgtcatcattggaggctttagcaagcaagaagagaggcagtggtggtggtggtggaggaggagctagctagcctgtgcttgctgatcggtgctgagctgaggaatcgttcgatcgatcgggcgagtcgacgaggggaagagttgagctgaggcgcatcgagaacaagatcaacaggcaggtcaccttctccaagcgccgcaacggcctcctcaagaaggcctacgagctgtccgttctctgcgacgccgaggtcgcgctcatcatcttctccagccgcggcaagctctacgagttcggcagcgccgggtataattaatacagacacaacaacacacacaaccaacaaaccagcatcaatttgaacctgcagatctgctgttttctctgatcaattgcttctttttttttgttcttttttgtttcttttatctgctgcaacggcgtcctgctcctctggggtttctcgttttctttttcatttatttttagcaggtgccaagtagccgagctactatacttacctggccatgttaattattttattccgtctgtctgtgtgtgtctgtgcatactactatagggacatggcgcggtgttcttataaaccgggaggccggatccctaactagcatgggaggatatcttttcagcggatctatacaaaccctactcctgctgacctctttcttccagtttctccgggtcttccttggattattattgcccatcttccgggttgtgcgtgtgtcagagacagctcgaacgataaatttctcaaaaccagtactagagagggtgtgttgtgtgtgagaactgagtggagagttagcatgaaggctgcaaactagaaaggaaggtatgttctttcctttttgatccatcaggggagccccttctggtattaagatctttccggcacattgattttcatactttgtgatgaccctggaagaatcggcgtagcagcgtagcaccgctccattttggtcttaccctcacctccccatgctatgaactgatcaatttcattgttcttcatcacccttctcctagctttccacttccttcggatctcatgccatgtttctcagcatgaatcaaatttaattcgtgttttctacttccatatatactggaagaaatttaattagatctatttttgctcgggaggtcttcatactttgagttctgatgccatcaccttatttcccccccccccttctcttgttctatcttcttcctcatcttggcttgatcattttgatctgtcagttatagcatgatgcattctcaatttgactgtatgtaagttcaaccggaaatatgttgaatggattttctatatatcaacacttgatgtcaggcctgcatctgtttcgcttgtggtggtgtggccaaaattgtctatatttgatctttgctcttctttctcctcatttcatgacgattcctactacggcttaaaccattctttattctttactaatcatggatgttgcttgactcctagttgtttcgtactagctcaacttggagatcttttcattatttgcctagttggtgggtacgtttgtgacagatctaaaatggtgcacgaaaagttttacttattatgaaaaaagggagcttaacagggtaatttctctatttattcgtgatgacattttttccttgataagggggattttttataatctgcactcacatgtttatatgtaaaatctagctcttttgttttgtttttggcatatttcccgctaagtatagagtttatgtggataacattataacttttcaagatccaatccacatctttgattgtgaaaatcatacaatagggaaaatcaactgaagggttaattagatgctatatgcatatatatatatatgtgcgcgcgcgcgcgcctgaatttaactatgtatgcatccaactgtttcattgaaaaagatttgatatttttcagtctattctttttcgagtatatatctaatatgtttcaatctgttttgaccattataagataaagcctatattcaccaggcatttgagatgatcttttcatgcatgaaaaagctgttgttatcacttcaactaaccagacgatctaacatgtatttgtataagaaacagaccttgatttccttctgtaaaatcatgcatgtgttcgttttgaattggagtcggcgcgcctgtgttttgaccgtcaggaaagtcttttttttccctgaatagtcaagggtctatacttcttgaagcaattgggacactaatcaattattgtttatacctcggaccatcttttccttcttcacaccactaatcagtttatgccttggaccattaattgtgttgttcacaagcttcttgtttatggtttacaaagcattcgcctagatttgtgtgtgtctctacacatcgatcacttttaaatacttgtcgctttcagttattcttttaacgtttggttatttatcttatttaaaaaaattatcgtattattatttattttgtttgtgatttactttattatcaaaagtatttcaaatatgacttatctttttttataagtgcactaatttttcaaataagatgaatggtcaaatgttacaagaaaaagttaaagcaaccactaatttagggcggaggtagtaaaacctagttattgtaaccaataattttatcaatctataaatgcaacacaaagtaacttcgtgatatctaacacaaagccacttcaacgatgaaagctgactgcatgttttatcaaaacacatgtgatcagtttgttggatgaaaaaaattatctatgtcataaatcaagagttataatataagcttctggctctacaagtaacatttctatgtttttttttacgttcttacatactatgttttgccaaaaaaaacatgatcattttgttggacgaaaagaaatagtaaatatagagtgacctttgatatcattataatataagcttctgcctctataaataacatctatgcactttttacgtcgtagtaatttgatatatgagaaatttacatataacatttttgtgcagcataaccaccatggatttgagcaatagctcacctgtcatcaccgatccggtggcgatcagccagcagttgttgggcggcctgccttcaaatctgatgcagttttcagtcatgcccggtggctactccagctctggcatgaacgttggtgtcagtaggctcaaaatcgaggaagtccttgtcaatggactgcttgatgccatgaaatcctcgtcacctcgcaggaggctgaatgtagcatttggcgaggacaattcatctgaagaagaagaccctgcttacagcgcttggatggcaaaatgtccttctgctttggcttccttcaagcaaattgtagccagtgcacaagggaagaagattgctgtgtttctagactatgacggcacactgtcgcctattgtggatgatcctgacaaagcagtgatgtctcccgtgatgagagctgctgtgagaaatgttgcgaagtacttccccactgcaattgtcagcggaaggtcccgcaataaggtgtttgaatttgtaaaactgaaggagctttattatgctggaagtcatggtatggacataatggcaccttcagcaaatcatgagcacagtgctgaaaagagcaaacaggccaatctcttccaacctgcacacgactttctgccaatgatcgatgaggttaccaagtccctcttgcaagttgtcagtggaattgaaggtgcaactgttgagaacaacaaattctgcgtttctgtagattatcgcaacgttgcagagaaggattggaaactggtcgcacggctcgtaaacgaagtgctggaggcttttcctcgtctcaaagtaaccaatggacgaatggttttagaggttcgtccggtgatcgactgggacaagggaaaggctgtggagtttctgctccagtcactcgggctaaatgactctgaaaatgtgatccccatctacattggagacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtgagctcgctaagcagccatcgatcagctgtcagaagttggagctaataataaaagggatgtggagtgggctacatgtatctcggatctctctgcgagccacctaatggtcttgcgtggccctttaatctgtatgtttttgtgtgtaagctactgctagctgtttgcaccttctgcgtccgtggttgtgtttccgtgctacctttttatgttttgatttggatcttgtttgaaaataatcttaccagctttgggtaaactgtttattacgtactctatatagcatatgtgaccgacgacaacggtttcattttagatgatgtgtatggatgatttctttccaaaatcacatctttagtataagagcaattttaccatccaataccaaattttatactagaaaatattttgggatatcaaaatttatggtacctccagtaccaaatgttgaatggtaaactttcataatatacaagtcactctaggatatttaagacaatttttagttttttcttattgttgcccttgttaaatacatgagaaattttacatcacttaaaatgtatcaagaggtatcaaatttttttaatacaaaatttagtactttctccgtttatatatgaatgtggacaatgcttgaaagtcttataacctgaaactgaggtagtgtatcgagaagtacaaaattttacactaaaatcccagtacttactcaataactgtaaaattactctaaatatgtactccctctatttcagattataagtcgttttaactttagtcaaagttaaactgtttcaagtttaaccaagtttgtagataaaagtagtaacatattcaacacaagacaaatatattataaaaacatattgaattatagatttaattaaattaatttggtattgcaagtattactaaatttgtttataaatttggtcgaatttaaaatagtttgactttaaccaaagtcaaaacaaattataatctaaaacaaaggtaatacattgtatcactctcatgaatggattgtaacatacattaatttaattactattttagttcttgtgcaaaagttgaaaacgatttatgtttggaatctttttgtggtgtatatatatgaaaccattcctctaccatccttccccaaccataatcctcacaaccgttagccccattgtgatctcacccagttgctagcctcttttgtcaccttgtcacagctctcctccattcattacacaatggcatcggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgaactagtgatatcggcgccatgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagt

SEQ ID NO: 8 - DNA, 12740 bp, Artificial Sequence
  Heterologous insertion sequence from 15777, plus the 5′ and 3′ flanking sequences.
gcacaacataattaaagttgataccttatgatattcgtattagatgtatttgaggttaaattattattactggtagtattaacagaaaatatattaatttaaaaggtgtaaagctataaaagtataaaggaatatgttaattatagtttttcttgaaaggatgaaccatgaaaatttagttgcgatgtttccatgtagtgaaccgagatgtcttatcagtaagtaaataggttaacatcaagaaagcacatttaataaaaaattggaaaggggacatacattaattaactatctaaaagtagtacacagaattagacatttagacataatatggtttgctaaatacgtagtaatctatataaacaaaattaaagttgtgtatttctaagacatgcatacgtacacaaacagaacatattgaaactagcctttgttgaacaatttttaacagcaaatgtggttttggtttgttaatattgcaagttattatgagtattcaacctagtaaattgaataatccttgcctgatatattgaatctatttgtatatatctagctagatacaattattagaacatgcttttattgtctacatatgtacccatgtaacatgaatagaaaataaataggtatgcttatcgtagagtatctatgaccatgtagcaagaaaaataagttaatatagagataaatcaatgggcaaaaaataaaatttgggggaaagaatctcactgctactactcttccactacaaagaacaacaaggttggttccaattccacttccggaacaatcagcgcaattgctccatccatccacagctgccaccgagactaggtgacacgatccaagccgccatggcgtcggctaagcctctacgctctagactgtgctggtggcactagggagactacgaaggttcacctgcaccttgaaggactagttgaggaaggtgttggcgaggatgttgaggggtttggacgatgaagcaccgtctagggaacaattgttctggcccgtgacgaggtgctgcaacgccatctccgccgccggagagacgtgcacgctctctgacgtcacatgtgagaggctatggttgtagcttcaatcgttggcggtgtgggctggtccggctcgatgtgaggggaggggagcgtgagttggagtcgtggaggaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggaccgctaggacgatggtgtgatgtgggaacacgaagaaaacatgaggaaaaaatattaaaatgaatttcccacttaaaatgcatcaaataaaaaaaataaagaaacgaccgggaatagacacagggtttgtgaactagctagggcaaacatcatatggtcccttgctgatgcacaagtacattgagatgtcatttcaattctgtgcatcatatgcatgtggtcccttgctgaatattactcttgaaatatctaccagtgccaatctattgcatgacttaattaattcacaggttttgttgattacattattagtaagcttgagagcacaagctcaatggatttttctataaatggggatcattttgcaattttctttgtcgtgcaaagttagccttctttattactacttctgtttttaaatatacgatcctattgacttttggtcatatatttaaccatgtatcttatttagatagtttgcgcaaatatatataccttcaatgataaaattagttacaatgaaacaaatgatatttacgcaattctttttactaaacaagtcacaagaagtacctgcagcaatatatgttggaaccgtgcagtagatcgagcctagctacgcaaaaaaacaaaaagagaaaaaaagggaaaggaaaaacattaatcatgcatgagcagtatgcccggcaactggaatttgtcaaagatatggggagaggagaataatacaagtactactactacctagctctaccatgcatatgcacccaaaggcaaactggattattggataaagcacagatgctggcaaaacaatccttaagcctcccctccctgcttctttatttttgggcagcctctaccggacggtgccgtggtccattggaccagtaggtggcgacatacatggtttgggttaagtctaggagagcagtgtgtgtgcgcgcgcaagagagagagactgtgagtctgggagtagccctctcccctcctttggccatcttcctcgtgtatatgcatatatgcatcatcgcaacggtgtatatttgtggtgtggcgggtgtggcattggattgcccccattttggctcgtgcttcccagttagggtaaaacctgtggtaaacttgctagccccacgccaaagttacccttctttattgttgaaagggagaggaggtgtgtgaattgtgatggagggagagagagagagatagaaagagagatgtgtgtcaaagcaagcaagaaaccagtttcacaaagagctactactagtactagtgtactactgtggtacagtgcccaatgtcctttctccggactcgactccactaatattctcctcttctcgcgcggctcgttatattctcgtcatcattggaggctttagcaagcaagaagagaggcagtggtggtggtggtggaggaggagctagctagcctgtgcttgctgatcggtgctgagctgaggaatcgttcgatcgatcgggcgagtcgacgaggggaagagttgagctgaggcgcatcgagaacaagatcaacaggcaggtcaccttctccaagcgccgcaacggcctcctcaagaaggcctacgagctgtccgttctctgcgacgccgaggtcgcgctcatcatcttctccagccgcggcaagctctacgagttcggcagcgccgggtataattaatacagacacaacaacacacacaaccaacaaaccagcatcaatttgaacctgcagatctgctgttttctctgatcaattgcttctttttttttgttcttttttgtttcttttatctgctgcaacggcgtcctgctcctctggggtttctcgttttctttttcatttatttttagcaggtgccaagtagccgagctactatacttacctggccatgttaattattttattccgtctgtctgtgtgtgtctgtgcatactactatagggacatggcgcggtgttcttataaaccgggaggccggatccctaactagcatgggaggatatcttttcagcggatctatacaaaccctactcctgctgacctctttcttccagtttctccgggtcttccttggattattattgcccatcttccgggttgtgcgtgtgtcagagacagctcgaacgataaatttctcaaaaccagtactagagagggtgtgttgtgtgtgagaactgagtggagagttagcatgaaggctgcaaactagaaaggaaggtatgttctttcctttttgatccatcaggggagccccttctggtattaagatctttccggcacattgattttcatactttgtgatgaccctggaagaatcggcgtagcagcgtagcaccgctccattttggtcttaccctcacctccccatgctatgaactgatcaatttcattgttcttcatcacccttctcctagctttccacttccttcggatctcatgccatgtttctcagcatgaatcaaatttaattcgtgttttctacttccatatatactggaagaaatttaattagatctatttttgctcgggaggtcttcatactttgagttctgatgccatcaccttatttcccccccccccttctcttgttctatcttcttcctcatcttggcttgatcattttgatctgtcagttatagcatgatgcattctcaatttgactgtatgtaagttcaaccggaaatatgttgaatggattttctatatatcaacacttgatgtcaggcctgcatctgtttcgcttgtggtggtgtggccaaaattgtctatatttgatctttgctcttctttctcctcatttcatgacgattcctactacggcttaaaccattctttattctttactaatcatggatgttgcttgactcctagttgtttcgtactagctcaacttggagatcttttcattatttgcctagttggtgggtacgtttgtgacagatctaaaatggtgcacgaaaagttttacttattatgaaaaaagggagcttaacagggtaatttctctatttattcgtgatgacattttttccttgataagggggattttttataatctgcactcacatgtttatatgtaaaatctagctcttttgttttgtttttggcatatttcccgctaagtatagagtttatgtggataacattataacttttcaagatccaatccacatctttgattgtgaaaatcatacaatagggaaaatcaactgaagggttaattagatgctatatgcatatatatatatatgtgcgcgcgcgcgcgcctgaatttaactatgtatgcatccaactgtttcattgaaaaagatttgatatttttcagtctattctttttcgagtatatatctaatatgtttcaatctgttttgaccattataagataaagcctatattcaccaggcatttgagatgatcttttcatgcatgaaaaagctgttgttatcacttcaactaaccagacgatctaacatgtatttgtataagaaacagaccttgatttccttctgtaaaatcatgcatgtgttcgttttgaattggagtcggcgcgcctgtgttttgaccgtcaggaaagtcttttttttccctgaatagtcaagggtctatacttcttgaagcaattgggacactaatcaattattgtttatacctcggaccatcttttccttcttcacaccactaatcagtttatgccttggaccattaattgtgttgttcacaagcttcttgtttatggtttacaaagcattcgcctagatttgtgtgtgtctctacacatcgatcacttttaaatacttgtcgctttcagttattcttttaacgtttggttatttatcttatttaaaaaaattatcgtattattatttattttgtttgtgatttactttattatcaaaagtatttcaaatatgacttatctttttttataagtgcactaatttttcaaataagatgaatggtcaaatgttacaagaaaaagttaaagcaaccactaatttagggcggaggtagtaaaacctagttattgtaaccaataattttatcaatctataaatgcaacacaaagtaacttcgtgatatctaacacaaagccacttcaacgatgaaagctgactgcatgttttatcaaaacacatgtgatcagtttgttggatgaaaaaaattatctatgtcataaatcaagagttataatataagcttctggctctacaagtaacatttctatgtttttttttacgttcttacatactatgttttgccaaaaaaaacatgatcattttgttggacgaaaagaaatagtaaatatagagtgacctttgatatcattataatataagcttctgcctctataaataacatctatgcactttttacgtcgtagtaatttgatatatgagaaatttacatataacatttttgtgcagcataaccaccatggatttgagcaatagctcacctgtcatcaccgatccggtggcgatcagccagcagttgttgggcggcctgccttcaaatctgatgcagttttcagtcatgcccggtggctactccagctctggcatgaacgttggtgtcagtaggctcaaaatcgaggaagtccttgtcaatggactgcttgatgccatgaaatcctcgtcacctcgcaggaggctgaatgtagcatttggcgaggacaattcatctgaagaagaagaccctgcttacagcgcttggatggcaaaatgtccttctgctttggcttccttcaagcaaattgtagccagtgcacaagggaagaagattgctgtgtttctagactatgacggcacactgtcgcctattgtggatgatcctgacaaagcagtgatgtctcccgtgatgagagctgctgtgagaaatgttgcgaagtacttccccactgcaattgtcagcggaaggtcccgcaataaggtgtttgaatttgtaaaactgaaggagctttattatgctggaagtcatggtatggacataatggcaccttcagcaaatcatgagcacagtgctgaaaagagcaaacaggccaatctcttccaacctgcacacgactttctgccaatgatcgatgaggttaccaagtccctcttgcaagttgtcagtggaattgaaggtgcaactgttgagaacaacaaattctgcgtttctgtagattatcgcaacgttgcagagaaggattggaaactggtcgcacggctcgtaaacgaagtgctggaggcttttcctcgtctcaaagtaaccaatggacgaatggttttagaggttcgtccggtgatcgactgggacaagggaaaggctgtggagtttctgctccagtcactcgggctaaatgactctgaaaatgtgatccccatctacattggagacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtgagctcgctaagcagccatcgatcagctgtcagaagttggagctaataataaaagggatgtggagtgggctacatgtatctcggatctctctgcgagccacctaatggtcttgcgtggccctttaatctgtatgtttttgtgtgtaagctactgctagctgtttgcaccttctgcgtccgtggttgtgtttccgtgctacctttttatgttttgatttggatcttgtttgaaaataatcttaccagctttgggtaaactgtttattacgtactctatatagcatatgtgaccgacgacaacggtttcattttagatgatgtgtatggatgatttctttccaaaatcacatctttagtataagagcaattttaccatccaataccaaattttatactagaaaatattttgggatatcaaaatttatggtacctccagtaccaaatgttgaatggtaaactttcataatatacaagtcactctaggatatttaagacaatttttagttttttcttattgttgcccttgttaaatacatgagaaattttacatcacttaaaatgtatcaagaggtatcaaatttttttaatacaaaatttagtactttctccgtttatatatgaatgtggacaatgcttgaaagtcttataacctgaaactgaggtagtgtatcgagaagtacaaaattttacactaaaatcccagtacttactcaataactgtaaaattactctaaatatgtactccctctatttcagattataagtcgttttaactttagtcaaagttaaactgtttcaagtttaaccaagtttgtagataaaagtagtaacatattcaacacaagacaaatatattataaaaacatattgaattatagatttaattaaattaatttggtattgcaagtattactaaatttgtttataaatttggtcgaatttaaaatagtttgactttaaccaaagtcaaaacaaattataatctaaaacaaaggtaatacattgtatcactctcatgaatggattgtaacatacattaatttaattactattttagttcttgtgcaaaagttgaaaacgatttatgtttggaatctttttgtggtgtatatatatgaaaccattcctctaccatccttccccaaccataatcctcacaaccgttagccccattgtgatctcacccagttgctagcctcttttgtcaccttgtcacagctctcctccattcattacacaatggcatcggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgaactagtgatatcggcgccatgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagtcggggaggggagcgtgagttgtagtcgcggtgtgatcttgacccgacgcatgatttaacaggaggtgactcgtgaaccttggacgacgatcgtgtggctgggaaaccaagggatgatatggacgcctcgagtgacgagatttgctcgtagctctattgatagcaggggcgaagctacgacttgttgatggtgtcaaatggcaccgataagttttagaaaatacaatgtaaaatattgtttacaatgttgatggcattcctataaaactgtgctgacactagcaaaacttaaggctggcttcgccccttattgatagtaaggctatggcctggagatcatgtatataatcgtttaaaatattattttatattatagactaagttatgtatatagtaaaataaaatttaaaatagaagatggaacgagcaaggagataacctaatgtgctgagacatgagagcgttatgccggaccgggccagaatcttcgcgcctccccggtccacagaaaccgcggtccgccagagcgcggcgcgcccagcgtccctgctcccaaagtcccatgcggcatgcgcccggcccaattggaaaataaagcaccgccgcatatcccgccctttccttttgtcggatggcgcgtcacggcgtttttccccataaaaaattcagaaatatataaacaccagacatgcgcccgcgcgctcttccctccctgccgcccgcgtcctcctctccacaccaatgcggccaccaccgcacgtcgccgcctccacctcgcctgccctccacgccgggtcgtcccggcgccggcctcaccaccgcggccggacgatgatgtccgtctcctgctgccacttggcggcgctcgtgtccatcctcgcggcgggcgcggctacggcgttgctcaccttgtccctaccctcttctcctggcgcgtccacgacgaggagaaccgacttcgttggagcattgtcagtggcaaacgagaccccgccaccgccgcacttgtcagcgcctccggcacccgccacgccgccccctgctccaccttccccgcccgccgacaggcctcgaaagcgagaggtggggtgggaccgagttgggttgggtgtcctgtgtcgtggtttcctc

SEQ ID NO: 9 - DNA, 4444 bp, Oryza sativa
  promoter (1)..(4444)
cggaccgctaggacgatggtgtgatgtgggaacacgaagaaaacatgaggaaaaaatattaaaatgaatttcccacttaaaatgcatcaaataaaaaaaataaagaaacgaccgggaatagacacagggtttgtgaactagctagggcaaacatcatatggtcccttgctgatgcacaagtacattgagatgtcatttcaattctgtgcatcatatgcatgtggtcccttgctgaatattactcttgaaatatctaccagtgccaatctattgcatgacttaattaattcacaggttttgttgattacattattagtaagcttgagagcacaagctcaatggatttttctataaatggggatcattttgcaattttctttgtcgtgcaaagttagccttctttattactacttctgtttttaaatatacgatcctattgacttttggtcatatatttaaccatgtatcttatttagatagtttgcgcaaatatatataccttcaatgataaaattagttacaatgaaacaaatgatatttacgcaattctttttactaaacaagtcacaagaagtacctgcagcaatatatgttggaaccgtgcagtagatcgagcctagctacgcaaaaaaacaaaaagagaaaaaaagggaaaggaaaaacattaatcatgcatgagcagtatgcccggcaactggaatttgtcaaagatatggggagaggagaataatacaagtactactactacctagctctaccatgcatatgcacccaaaggcaaactggattattggataaagcacagatgctggcaaaacaatccttaagcctcccctccctgcttctttatttttgggcagcctctaccggacggtgccgtggtccattggaccagtaggtggcgacatacatggtttgggttaagtctaggagagcagtgtgtgtgcgcgcgcaagagagagagactgtgagtctgggagtagccctctcccctcctttggccatcttcctcgtgtatatgcatatatgcatcatcgcaacggtgtatatttgtggtgtggcgggtgtggcattggattgcccccattttggctcgtgcttcccagttagggtaaaacctgtggtaaacttgctagccccacgccaaagttacccttctttattgttgaaagggagaggaggtgtgtgaattgtgatggagggagagagagagagatagaaagagagatgtgtgtcaaagcaagcaagaaaccagtttcacaaagagctactactagtactagtgtactactgtggtacagtgcccaatgtcctttctccggactcgactccactaatattctcctcttctcgcgcggctcgttatattctcgtcatcattggaggctttagcaagcaagaagagaggcagtggtggtggtggtggaggaggagctagctagcctgtgcttgctgatcggtgctgagctgaggaatcgttcgatcgatcgggcgagtcgacgaggggaagagttgagctgaggcgcatcgagaacaagatcaacaggcaggtcaccttctccaagcgccgcaacggcctcctcaagaaggcctacgagctgtccgttctctgcgacgccgaggtcgcgctcatcatcttctccagccgcggcaagctctacgagttcggcagcgccgggtataattaatacagacacaacaacacacacaaccaacaaaccagcatcaatttgaacctgcagatctgctgttttctctgatcaattgcttctttttttttgttcttttttgtttcttttatctgctgcaacggcgtcctgctcctctggggtttctcgttttctttttcatttatttttagcaggtgccaagtagccgagctactatacttacctggccatgttaattattttattccgtctgtctgtgtgtgtctgtgcatactactatagggacatggcgcggtgttcttataaaccgggaggccggatccctaactagcatgggaggatatcttttcagcggatctatacaaaccctactcctgctgacctctttcttccagtttctccgggtcttccttggattattattgcccatcttccgggttgtgcgtgtgtcagagacagctcgaacgataaatttctcaaaaccagtactagagagggtgtgttgtgtgtgagaactgagtggagagttagcatgaaggctgcaaactagaaaggaaggtatgttctttcctttttgatccatcaggggagccccttctggtattaagatctttccggcacattgattttcatactttgtgatgaccctggaagaatcggcgtagcagcgtagcaccgctccattttggtcttaccctcacctccccatgctatgaactgatcaatttcattgttcttcatcacccttctcctagctttccacttccttcggatctcatgccatgtttctcagcatgaatcaaatttaattcgtgttttctacttccatatatactggaagaaatttaattagatctatttttgctcgggaggtcttcatactttgagttctgatgccatcaccttatttcccccccccccttctcttgttctatcttcttcctcatcttggcttgatcattttgatctgtcagttatagcatgatgcattctcaatttgactgtatgtaagttcaaccggaaatatgttgaatggattttctatatatcaacacttgatgtcaggcctgcatctgtttcgcttgtggtggtgtggccaaaattgtctatatttgatctttgctcttctttctcctcatttcatgacgattcctactacggcttaaaccattctttattctttactaatcatggatgttgcttgactcctagttgtttcgtactagctcaacttggagatcttttcattatttgcctagttggtgggtacgtttgtgacagatctaaaatggtgcacgaaaagttttacttattatgaaaaaagggagcttaacagggtaatttctctatttattcgtgatgacattttttccttgataagggggattttttataatctgcactcacatgtttatatgtaaaatctagctcttttgttttgtttttggcatatttcccgctaagtatagagtttatgtggataacattataacttttcaagatccaatccacatctttgattgtgaaaatcatacaatagggaaaatcaactgaagggttaattagatgctatatgcatatatatatatatgtgcgcgcgcgcgcgcctgaatttaactatgtatgcatccaactgtttcattgaaaaagatttgatatttttcagtctattctttttcgagtatatatctaatatgtttcaatctgttttgaccattataagataaagcctatattcaccaggcatttgagatgatcttttcatgcatgaaaaagctgttgttatcacttcaactaaccagacgatctaacatgtatttgtataagaaacagaccttgatttccttctgtaaaatcatgcatgtgttcgttttgaattggagtcggcgcgcctgtgttttgaccgtcaggaaagtcttttttttccctgaatagtcaagggtctatacttcttgaagcaattgggacactaatcaattattgtttatacctcggaccatcttttccttcttcacaccactaatcagtttatgccttggaccattaattgtgttgttcacaagcttcttgtttatggtttacaaagcattcgcctagatttgtgtgtgtctctacacatcgatcacttttaaatacttgtcgctttcagttattcttttaacgtttggttatttatcttatttaaaaaaattatcgtattattatttattttgtttgtgatttactttattatcaaaagtatttcaaatatgacttatctttttttataagtgcactaatttttcaaataagatgaatggtcaaatgttacaagaaaaagttaaagcaaccactaatttagggcggaggtagtaaaacctagttattgtaaccaataattttatcaatctataaatgcaacacaaagtaacttcgtgatatctaacacaaagccacttcaacgatgaaagctgactgcatgttttatcaaaacacatgtgatcagtttgttggatgaaaaaaattatctatgtcataaatcaagagttataatataagcttctggctctacaagtaacatttctatgtttttttttacgttcttacatactatgttttgccaaaaaaaacatgatcattttgttggacgaaaagaaatagtaaatatagagtgacctttgatatcattataatataagcttctgcctctataaataacatctatgcactttttacgtcgtagtaatttgatatatgagaaatttacatataacatttttgtgcagcataaccac

SEQ ID NO: 10 - DNA, 1116 bp, Oryza sativa
  gene (1)..(1116)
atggatttgagcaatagctcacctgtcatcaccgatccggtggcgatcagccagcagttgttgggcggcctgccttcaaatctgatgcagttttcagtcatgcccggtggctactccagctctggcatgaacgttggtgtcagtaggctcaaaatcgaggaagtccttgtcaatggactgcttgatgccatgaaatcctcgtcacctcgcaggaggctgaatgtagcatttggcgaggacaattcatctgaagaagaagaccctgcttacagcgcttggatggcaaaatgtccttctgctttggcttccttcaagcaaattgtagccagtgcacaagggaagaagattgctgtgtttctagactatgacggcacactgtcgcctattgtggatgatcctgacaaagcagtgatgtctcccgtgatgagagctgctgtgagaaatgttgcgaagtacttccccactgcaattgtcagcggaaggtcccgcaataaggtgtttgaatttgtaaaactgaaggagctttattatgctggaagtcatggtatggacataatggcaccttcagcaaatcatgagcacagtgctgaaaagagcaaacaggccaatctcttccaacctgcacacgactttctgccaatgatcgatgaggttaccaagtccctcttgcaagttgtcagtggaattgaaggtgcaactgttgagaacaacaaattctgcgtttctgtagattatcgcaacgttgcagagaaggattggaaactggtcgcacggctcgtaaacgaagtgctggaggcttttcctcgtctcaaagtaaccaatggacgaatggttttagaggttcgtccggtgatcgactgggacaagggaaaggctgtggagtttctgctccagtcactcgggctaaatgactctgaaaatgtgatccccatctacattggagacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtga

SEQ ID NO: 11 - protein, 371 aa, Oryza sativa
MDLSNSSPVITDPVAISQQLLGGLPSNLMQFSVMPGGYSSSGMNVGVSRLKIEEVLVNGLLDAMKSSSPRRRLNVAFGEDNSSEEEDPAYSAWMAKCPSALASFKQIVASAQGKKIAVFLDYDGTLSPIVDDPDKAVMSPVMRAAVRNVAKYFPTAIVSGRSRNKVFEFVKLKELYYAGSHGMDIMAPSANHEHSAEKSKQANLFQPAHDFLPMIDEVTKSLLQVVSGIEGATVENNKFCVSVDYRNVAEKDWKLVARLVNEVLEAFPRLKVTNGRMVLEVRPVIDWDKGKAVEFLLQSLGLNDSENVIPIYIGDDRTDEDAFKVLRQRNCGYGILVSQVPKETEAFYSLRDPSEVMEFLNFLVRWKKHSV

SEQ ID NO: 12 - DNA, 26 bp, Zea mays
cttgaaaggatgaaccatgaaaattt

SEQ ID NO: 13 - DNA, 26 bp, Zea mays
cgtgactcccttaattctccgctcat

SEQ ID NO: 14 - DNA, 21 bp, Zea mays
cctgttgccggtcttgcgatg

SEQ ID NO: 15 - DNA, 21 bp, Zea mays
ccaccccacctctcgctttcg

SEQ ID NO: 16 - DNA, 27 bp, Zea mays
agttgcgatgtttccatgtagtgaacc

SEQ ID NO: 17 - DNA, 20 bp, Zea mays
ccggaggcgctgacaagtgc

SEQ ID NO: 18 - DNA, 30 bp, Zea mays
tgatcaagccaagatgaggaagaagataga

SEQ ID NO: 19 - DNA, 30 bp, Zea mays
agttagcatgaaggctgcaaactagaaagg

SEQ ID NO: 20 - DNA, 30 bp, Zea mays
aaatcgttttcaacttttgcacaagaacta

SEQ ID NO: 21 - DNA, 26 bp, Zea mays
agacgacagaactgacgaagacgctt

SEQ ID NO: 22 - DNA, 25 bp, Zea mays
gatcacaccgcgactacaactcacg

SEQ ID NO: 23 - DNA, 1095 bp, Zea mays
cttgaaaggatgaaccatgaaaatttagttgcgatgtttccatgtagtgaaccgagatgtcttatcagtaagtaaataggttaacatcaagaaagcacatttaataaaaaattggaaaggggacatacattaattaactatctaaaagtagtacacagaattagacatttagacataatatggtttgctaaatacgtagtaatctatataaacaaaattaaagttgtgtatttctaagacatgcatacgtacacaaacagaacatattgaaactagcctttgttgaacaatttttaacagcaaatgtggttttggtttgttaatattgcaagttattatgagtattcaacctagtaaattgaataatccttgcctgatatattgaatctatttgtatatatctagctagatacaattattagaacatgcttttattgtctacatatgtacccatgtaacatgaatagaaaataaataggtatgcttatcgtagagtatctatgaccatgtagcaagaaaaataagttaatatagagataaatcaatgggcaaaaaataaaatttgggggaaagaatctcactgctactactcttccactacaaagaacaacaaggttggttccaattccacttccggaacaatcagcgcaattgctccatccatccacagctgccaccgagactaggtgacacgatccaagccgccatggcgtcggctaagcctctacgctctagactgtgctggtggcactagggagactacgaaggttcacctgcaccttgaaggactagttgaggaaggtgttggcgaggatgttgaggggtttggacgatgaagcaccgtctagggaacaattgttctggcccgtgacgaggtgctgcaacgccatctccgccgccggagagacgtgcacgctctctgacgtcacatgtgagaggctatggttgtagcttcaatcgttggcggtgtgggctggtccggctcgatgtgaggggaggggagcgtgagttggagtcgtggaggaaacactgatagtttaaactgaaggcgggaaacgacaatctgatcatgagcggagaattaagggagtcacg

SEQ ID NO: 24 - DNA, 1373 bp, Zea mays
cctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagtcggggaggggagcgtgagttgtagtcgcggtgtgatcttgacccgacgcatgatttaacaggaggtgactcgtgaaccttggacgacgatcgtgtggctgggaaaccaagggatgatatggacgcctcgagtgacgagatttgctcgtagctctattgatagcaggggcgaagctacgacttgttgatggtgtcaaatggcaccgataagttttagaaaatacaatgtaaaatattgtttacaatgttgatggcattcctataaaactgtgctgacactagcaaaacttaaggctggcttcgccccttattgatagtaaggctatggcctggagatcatgtatataatcgtttaaaatattattttatattatagactaagttatgtatatagtaaaataaaatttaaaatagaagatggaacgagcaaggagataacctaatgtgctgagacatgagagcgttatgccggaccgggccagaatcttcgcgcctccccggtccacagaaaccgcggtccgccagagcgcggcgcgcccagcgtccctgctcccaaagtcccatgcggcatgcgcccggcccaattggaaaataaagcaccgccgcatatcccgccctttccttttgtcggatggcgcgtcacggcgtttttccccataaaaaattcagaaatatataaacaccagacatgcgcccgcgcgctcttccctccctgccgcccgcgtcctcctctccacaccaatgcggccaccaccgcacgtcgccgcctccacctcgcctgccctccacgccgggtcgtcccggcgccggcctcaccaccgcggccggacgatgatgtccgtctcctgctgccacttggcggcgctcgtgtccatcctcgcggcgggcgcggctacggcgttgctcaccttgtccctaccctcttctcctggcgcgtccacgacgaggagaaccgacttcgttggagcattgtcagtggcaaacgagaccccgccaccgccgcacttgtcagcgcctccggcacccgccacgccgccccctgctccaccttccccgcccgccgacaggcctcgaaagcgagaggtggggtgg

SEQ ID NO: 25 - DNA, 12453 bp, Zea mays
agttgcgatgtttccatgtagtgaaccgagatgtcttatcagtaagtaaataggttaacatcaagaaagcacatttaataaaaaattggaaaggggacatacattaattaactatctaaaagtagtacacagaattagacatttagacataatatggtttgctaaatacgtagtaatctatataaacaaaattaaagttgtgtatttctaagacatgcatacgtacacaaacagaacatattgaaactagcctttgttgaacaatttttaacagcaaatgtggttttggtttgttaatattgcaagttattatgagtattcaacctagtaaattgaataatccttgcctgatatattgaatctatttgtatatatctagctagatacaattattagaacatgcttttattgtctacatatgtacccatgtaacatgaatagaaaataaataggtatgcttatcgtagagtatctatgaccatgtagcaagaaaaataagttaatatagagataaatcaatgggcaaaaaataaaatttgggggaaagaatctcactgctactactcttccactacaaagaacaacaaggttggttccaattccacttccggaacaatcagcgcaattgctccatccatccacagctgccaccgagactaggtgacacgatccaagccgccatggcgtcggctaagcctctacgctctagactgtgctggtggcactagggagactacgaaggttcacctgcaccttgaaggactagttgaggaaggtgttggcgaggatgttgaggggtttggacgatgaagcaccgtctagggaacaattgttctggcccgtgacgaggtgctgcaacgccatctccgccgccggagagacgtgcacgctctctgacgtcacatgtgagaggctatggttgtagcttcaatcgttggcggtgtgggctggtccggctcgatgtgaggggaggggagcgtgagttggagtcgtggaggaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggaccgctaggacgatggtgtgatgtgggaacacgaagaaaacatgaggaaaaaatattaaaatgaatttcccacttaaaatgcatcaaataaaaaaaataaagaaacgaccgggaatagacacagggtttgtgaactagctagggcaaacatcatatggtcccttgctgatgcacaagtacattgagatgtcatttcaattctgtgcatcatatgcatgtggtcccttgctgaatattactcttgaaatatctaccagtgccaatctattgcatgacttaattaattcacaggttttgttgattacattattagtaagcttgagagcacaagctcaatggatttttctataaatggggatcattttgcaattttctttgtcgtgcaaagttagccttctttattactacttctgtttttaaatatacgatcctattgacttttggtcatatatttaaccatgtatcttatttagatagtttgcgcaaatatatataccttcaatgataaaattagttacaatgaaacaaatgatatttacgcaattctttttactaaacaagtcacaagaagtacctgcagcaatatatgttggaaccgtgcagtagatcgagcctagctacgcaaaaaaacaaaaagagaaaaaaagggaaaggaaaaacattaatcatgcatgagcagtatgcccggcaactggaatttgtcaaagatatggggagaggagaataatacaagtactactactacctagctctaccatgcatatgcacccaaaggcaaactggattattggataaagcacagatgctggcaaaacaatccttaagcctcccctccctgcttctttatttttgggcagcctctaccggacggtgccgtggtccattggaccagtaggtggcgacatacatggtttgggttaagtctaggagagcagtgtgtgtgcgcgcgcaagagagagagactgtgagtctgggagtagccctctcccctcctttggccatcttcctcgtgtatatgcatatatgcatcatcgcaacggtgtatatttgtggtgtggcgggtgtggcattggattgcccccattttggctcgtgcttcccagttagggtaaaacctgtggtaaacttgctagccccacgccaaagttacccttctttattgttgaaagggagaggaggtgtgtgaattgtgatggagggagagagagagagatagaaagagagatgtgtgtcaaagcaagcaagaaaccagtttcacaaagagctactactagtactagtgtactactgtggtacagtgcccaatgtcctttctccggactcgactccactaatattctcctcttctcgcgcggctcgttatattctcgtcatcattggaggctttagcaagcaagaagagaggcagtggtggtggtggtggaggaggagctagctagcctgtgcttgctgatcggtgctgagctgaggaatcgttcgatcgatcgggcgagtcgacgaggggaagagttgagctgaggcgcatcgagaacaagatcaacaggcaggtcaccttctccaagcgccgcaacggcctcctcaagaaggcctacgagctgtccgttctctgcgacgccgaggtcgcgctcatcatcttctccagccgcggcaagctctacgagttcggcagcgccgggtataattaatacagacacaacaacacacacaaccaacaaaccagcatcaatttgaacctgcagatctgctgttttctctgatcaattgcttctttttttttgttcttttttgtttcttttatctgctgcaacggcgtcctgctcctctggggtttctcgttttctttttcatttatttttagcaggtgccaagtagccgagctactatacttacctggccatgttaattattttattccgtctgtctgtgtgtgtctgtgcatactactatagggacatggcgcggtgttcttataaaccgggaggccggatccctaactagcatgggaggatatcttttcagcggatctatacaaaccctactcctgctgacctctttcttccagtttctccgggtcttccttggattattattgcccatcttccgggttgtgcgtgtgtcagagacagctcgaacgataaatttctcaaaaccagtactagagagggtgtgttgtgtgtgagaactgagtggagagttagcatgaaggctgcaaactagaaaggaaggtatgttctttcctttttgatccatcaggggagccccttctggtattaagatctttccggcacattgattttcatactttgtgatgaccctggaagaatcggcgtagcagcgtagcaccgctccattttggtcttaccctcacctccccatgctatgaactgatcaatttcattgttcttcatcacccttctcctagctttccacttccttcggatctcatgccatgtttctcagcatgaatcaaatttaattcgtgttttctacttccatatatactggaagaaatttaattagatctatttttgctcgggaggtcttcatactttgagttctgatgccatcaccttatttcccccccccccttctcttgttctatcttcttcctcatcttggcttgatcattttgatctgtcagttatagcatgatgcattctcaatttgactgtatgtaagttcaaccggaaatatgttgaatggattttctatatatcaacacttgatgtcaggcctgcatctgtttcgcttgtggtggtgtggccaaaattgtctatatttgatctttgctcttctttctcctcatttcatgacgattcctactacggcttaaaccattctttattctttactaatcatggatgttgcttgactcctagttgtttcgtactagctcaacttggagatcttttcattatttgcctagttggtgggtacgtttgtgacagatctaaaatggtgcacgaaaagttttacttattatgaaaaaagggagcttaacagggtaatttctctatttattcgtgatgacattttttccttgataagggggattttttataatctgcactcacatgtttatatgtaaaatctagctcttttgttttgtttttggcatatttcccgctaagtatagagtttatgtggataacattataacttttcaagatccaatccacatctttgattgtgaaaatcatacaatagggaaaatcaactgaagggttaattagatgctatatgcatatatatatatatgtgcgcgcgcgcgcgcctgaatttaactatgtatgcatccaactgtttcattgaaaaagatttgatatttttcagtctattctttttcgagtatatatctaatatgtttcaatctgttttgaccattataagataaagcctatattcaccaggcatttgagatgatcttttcatgcatgaaaaagctgttgttatcacttcaactaaccagacgatctaacatgtatttgtataagaaacagaccttgatttccttctgtaaaatcatgcatgtgttcgttttgaattggagtcggcgcgcctgtgttttgaccgtcaggaaagtcttttttttccctgaatagtcaagggtctatacttcttgaagcaattgggacactaatcaattattgtttatacctcggaccatcttttccttcttcacaccactaatcagtttatgccttggaccattaattgtgttgttcacaagcttcttgtttatggtttacaaagcattcgcctagatttgtgtgtgtctctacacatcgatcacttttaaatacttgtcgctttcagttattcttttaacgtttggttatttatcttatttaaaaaaattatcgtattattatttattttgtttgtgatttactttattatcaaaagtatttcaaatatgacttatctttttttataagtgcactaatttttcaaataagatgaatggtcaaatgttacaagaaaaagttaaagcaaccactaatttagggcggaggtagtaaaacctagttattgtaaccaataattttatcaatctataaatgcaacacaaagtaacttcgtgatatctaacacaaagccacttcaacgatgaaagctgactgcatgttttatcaaaacacatgtgatcagtttgttggatgaaaaaaattatctatgtcataaatcaagagttataatataagcttctggctctacaagtaacatttctatgtttttttttacgttcttacatactatgttttgccaaaaaaaacatgatcattttgttggacgaaaagaaatagtaaatatagagtgacctttgatatcattataatataagcttctgcctctataaataacatctatgcactttttacgtcgtagtaatttgatatatgagaaatttacatataacatttttgtgcagcataaccaccatggatttgagcaatagctcacctgtcatcaccgatccggtggcgatcagccagcagttgttgggcggcctgccttcaaatctgatgcagttttcagtcatgcccggtggctactccagctctggcatgaacgttggtgtcagtaggctcaaaatcgaggaagtccttgtcaatggactgcttgatgccatgaaatcctcgtcacctcgcaggaggctgaatgtagcatttggcgaggacaattcatctgaagaagaagaccctgcttacagcgcttggatggcaaaatgtccttctgctttggcttccttcaagcaaattgtagccagtgcacaagggaagaagattgctgtgtttctagactatgacggcacactgtcgcctattgtggatgatcctgacaaagcagtgatgtctcccgtgatgagagctgctgtgagaaatgttgcgaagtacttccccactgcaattgtcagcggaaggtcccgcaataaggtgtttgaatttgtaaaactgaaggagctttattatgctggaagtcatggtatggacataatggcaccttcagcaaatcatgagcacagtgctgaaaagagcaaacaggccaatctcttccaacctgcacacgactttctgccaatgatcgatgaggttaccaagtccctcttgcaagttgtcagtggaattgaaggtgcaactgttgagaacaacaaattctgcgtttctgtagattatcgcaacgttgcagagaaggattggaaactggtcgcacggctcgtaaacgaagtgctggaggcttttcctcgtctcaaagtaaccaatggacgaatggttttagaggttcgtccggtgatcgactgggacaagggaaaggctgtggagtttctgctccagtcactcgggctaaatgactctgaaaatgtgatccccatctacattggagacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtgagctcgctaagcagccatcgatcagctgtcagaagttggagctaataataaaagggatgtggagtgggctacatgtatctcggatctctctgcgagccacctaatggtcttgcgtggccctttaatctgtatgtttttgtgtgtaagctactgctagctgtttgcaccttctgcgtccgtggttgtgtttccgtgctacctttttatgttttgatttggatcttgtttgaaaataatcttaccagctttgggtaaactgtttattacgtactctatatagcatatgtgaccgacgacaacggtttcattttagatgatgtgtatggatgatttctttccaaaatcacatctttagtataagagcaattttaccatccaataccaaattttatactagaaaatattttgggatatcaaaatttatggtacctccagtaccaaatgttgaatggtaaactttcataatatacaagtcactctaggatatttaagacaatttttagttttttcttattgttgcccttgttaaatacatgagaaattttacatcacttaaaatgtatcaagaggtatcaaatttttttaatacaaaatttagtactttctccgtttatatatgaatgtggacaatgcttgaaagtcttataacctgaaactgaggtagtgtatcgagaagtacaaaattttacactaaaatcccagtacttactcaataactgtaaaattactctaaatatgtactccctctatttcagattataagtcgttttaactttagtcaaagttaaactgtttcaagtttaaccaagtttgtagataaaagtagtaacatattcaacacaagacaaatatattataaaaacatattgaattatagatttaattaaattaatttggtattgcaagtattactaaatttgtttataaatttggtcgaatttaaaatagtttgactttaaccaaagtcaaaacaaattataatctaaaacaaaggtaatacattgtatcactctcatgaatggattgtaacatacattaatttaattactattttagttcttgtgcaaaagttgaaaacgatttatgtttggaatctttttgtggtgtatatatatgaaaccattcctctaccatccttccccaaccataatcctcacaaccgttagccccattgtgatctcacccagttgctagcctcttttgtcaccttgtcacagctctcctccattcattacacaatggcatcggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgaactagtgatatcggcgccatgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagtcggggaggggagcgtgagttgtagtcgcggtgtgatcttgacccgacgcatgatttaacaggaggtgactcgtgaaccttggacgacgatcgtgtggctgggaaaccaagggatgatatggacgcctcgagtgacgagatttgctcgtagctctattgatagcaggggcgaagctacgacttgttgatggtgtcaaatggcaccgataagttttagaaaatacaatgtaaaatattgtttacaatgttgatggcattcctataaaactgtgctgacactagcaaaacttaaggctggcttcgccccttattgatagtaaggctatggcctggagatcatgtatataatcgtttaaaatattattttatattatagactaagttatgtatatagtaaaataaaatttaaaatagaagatggaacgagcaaggagataacctaatgtgctgagacatgagagcgttatgccggaccgggccagaatcttcgcgcctccccggtccacagaaaccgcggtccgccagagcgcggcgcgcccagcgtccctgctcccaaagtcccatgcggcatgcgcccggcccaattggaaaataaagcaccgccgcatatcccgccctttccttttgtcggatggcgcgtcacggcgtttttccccataaaaaattcagaaatatataaacaccagacatgcgcccgcgcgctcttccctccctgccgcccgcgtcctcctctccacaccaatgcggccaccaccgcacgtcgccgcctccacctcgcctgccctccacgccgggtcgtcccggcgccggcctcaccaccgcggccggacgatgatgtccgtctcctgctgccacttggcggcgctcgtgtccatcctcgcggcgggcgcggctacggcgttgctcaccttgtccctaccctcttctcctggcgcgtccacgacgaggagaaccgacttcgttggagcattgtcagtggcaaacgagaccccgccaccgccgcacttgtcagcgcctccgg

SEQ ID NO: 26 - DNA, 4307 bp, Zea mays
agttgcgatgtttccatgtagtgaaccgagatgtcttatcagtaagtaaataggttaacatcaagaaagcacatttaataaaaaattggaaaggggacatacattaattaactatctaaaagtagtacacagaattagacatttagacataatatggtttgctaaatacgtagtaatctatataaacaaaattaaagttgtgtatttctaagacatgcatacgtacacaaacagaacatattgaaactagcctttgttgaacaatttttaacagcaaatgtggttttggtttgttaatattgcaagttattatgagtattcaacctagtaaattgaataatccttgcctgatatattgaatctatttgtatatatctagctagatacaattattagaacatgcttttattgtctacatatgtacccatgtaacatgaatagaaaataaataggtatgcttatcgtagagtatctatgaccatgtagcaagaaaaataagttaatatagagataaatcaatgggcaaaaaataaaatttgggggaaagaatctcactgctactactcttccactacaaagaacaacaaggttggttccaattccacttccggaacaatcagcgcaattgctccatccatccacagctgccaccgagactaggtgacacgatccaagccgccatggcgtcggctaagcctctacgctctagactgtgctggtggcactagggagactacgaaggttcacctgcaccttgaaggactagttgaggaaggtgttggcgaggatgttgaggggtttggacgatgaagcaccgtctagggaacaattgttctggcccgtgacgaggtgctgcaacgccatctccgccgccggagagacgtgcacgctctctgacgtcacatgtgagaggctatggttgtagcttcaatcgttggcggtgtgggctggtccggctcgatgtgaggggaggggagcgtgagttggagtcgtggaggaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggaccgctaggacgatggtgtgatgtgggaacacgaagaaaacatgaggaaaaaatattaaaatgaatttcccacttaaaatgcatcaaataaaaaaaataaagaaacgaccgggaatagacacagggtttgtgaactagctagggcaaacatcatatggtcccttgctgatgcacaagtacattgagatgtcatttcaattctgtgcatcatatgcatgtggtcccttgctgaatattactcttgaaatatctaccagtgccaatctattgcatgacttaattaattcacaggttttgttgattacattattagtaagcttgagagcacaagctcaatggatttttctataaatggggatcattttgcaattttctttgtcgtgcaaagttagccttctttattactacttctgtttttaaatatacgatcctattgacttttggtcatatatttaaccatgtatcttatttagatagtttgcgcaaatatatataccttcaatgataaaattagttacaatgaaacaaatgatatttacgcaattctttttactaaacaagtcacaagaagtacctgcagcaatatatgttggaaccgtgcagtagatcgagcctagctacgcaaaaaaacaaaaagagaaaaaaagggaaaggaaaaacattaatcatgcatgagcagtatgcccggcaactggaatttgtcaaagatatggggagaggagaataatacaagtactactactacctagctctaccatgcatatgcacccaaaggcaaactggattattggataaagcacagatgctggcaaaacaatccttaagcctcccctccctgcttctttatttttgggcagcctctaccggacggtgccgtggtccattggaccagtaggtggcgacatacatggtttgggttaagtctaggagagcagtgtgtgtgcgcgcgcaagagagagagactgtgagtctgggagtagccctctcccctcctttggccatcttcctcgtgtatatgcatatatgcatcatcgcaacggtgtatatttgtggtgtggcgggtgtggcattggattgcccccattttggctcgtgcttcccagttagggtaaaacctgtggtaaacttgctagccccacgccaaagttacccttctttattgttgaaagggagaggaggtgtgtgaattgtgatggagggagagagagagagatagaaagagagatgtgtgtcaaagcaagcaagaaaccagtttcacaaagagctactactagtactagtgtactactgtggtacagtgcccaatgtcctttctccggactcgactccactaatattctcctcttctcgcgcggctcgttatattctcgtcatcattggaggctttagcaagcaagaagagaggcagtggtggtggtggtggaggaggagctagctagcctgtgcttgctgatcggtgctgagctgaggaatcgttcgatcgatcgggcgagtcgacgaggggaagagttgagctgaggcgcatcgagaacaagatcaacaggcaggtcaccttctccaagcgccgcaacggcctcctcaagaaggcctacgagctgtccgttctctgcgacgccgaggtcgcgctcatcatcttctccagccgcggcaagctctacgagttcggcagcgccgggtataattaatacagacacaacaacacacacaaccaacaaaccagcatcaatttgaacctgcagatctgctgttttctctgatcaattgcttctttttttttgttcttttttgtttcttttatctgctgcaacggcgtcctgctcctctggggtttctcgttttctttttcatttatttttagcaggtgccaagtagccgagctactatacttacctggccatgttaattattttattccgtctgtctgtgtgtgtctgtgcatactactatagggacatggcgcggtgttcttataaaccgggaggccggatccctaactagcatgggaggatatcttttcagcggatctatacaaaccctactcctgctgacctctttcttccagtttctccgggtcttccttggattattattgcccatcttccgggttgtgcgtgtgtcagagacagctcgaacgataaatttctcaaaaccagtactagagagggtgtgttgtgtgtgagaactgagtggagagttagcatgaaggctgcaaactagaaaggaaggtatgttctttcctttttgatccatcaggggagccccttctggtattaagatctttccggcacattgattttcatactttgtgatgaccctggaagaatcggcgtagcagcgtagcaccgctccattttggtcttaccctcacctccccatgctatgaactgatcaatttcattgttcttcatcacccttctcctagctttccacttccttcggatctcatgccatgtttctcagcatgaatcaaatttaattcgtgttttctacttccatatatactggaagaaatttaattagatctatttttgctcgggaggtcttcatactttgagttctgatgccatcaccttatttcccccccccccttctcttgttctatcttcttcctcatcttggcttgatca

SEQ ID NO: 27 - DNA, 4448 bp, Zea mays
agttagcatgaaggctgcaaactagaaaggaaggtatgttctttcctttttgatccatcaggggagccccttctggtattaagatctttccggcacattgattttcatactttgtgatgaccctggaagaatcggcgtagcagcgtagcaccgctccattttggtcttaccctcacctccccatgctatgaactgatcaatttcattgttcttcatcacccttctcctagctttccacttccttcggatctcatgccatgtttctcagcatgaatcaaatttaattcgtgttttctacttccatatatactggaagaaatttaattagatctatttttgctcgggaggtcttcatactttgagttctgatgccatcaccttatttcccccccccccttctcttgttctatcttcttcctcatcttggcttgatcattttgatctgtcagttatagcatgatgcattctcaatttgactgtatgtaagttcaaccggaaatatgttgaatggattttctatatatcaacacttgatgtcaggcctgcatctgtttcgcttgtggtggtgtggccaaaattgtctatatttgatctttgctcttctttctcctcatttcatgacgattcctactacggcttaaaccattctttattctttactaatcatggatgttgcttgactcctagttgtttcgtactagctcaacttggagatcttttcattatttgcctagttggtgggtacgtttgtgacagatctaaaatggtgcacgaaaagttttacttattatgaaaaaagggagcttaacagggtaatttctctatttattcgtgatgacattttttccttgataagggggattttttataatctgcactcacatgtttatatgtaaaatctagctcttttgttttgtttttggcatatttcccgctaagtatagagtttatgtggataacattataacttttcaagatccaatccacatctttgattgtgaaaatcatacaatagggaaaatcaactgaagggttaattagatgctatatgcatatatatatatatgtgcgcgcgcgcgcgcctgaatttaactatgtatgcatccaactgtttcattgaaaaagatttgatatttttcagtctattctttttcgagtatatatctaatatgtttcaatctgttttgaccattataagataaagcctatattcaccaggcatttgagatgatcttttcatgcatgaaaaagctgttgttatcacttcaactaaccagacgatctaacatgtatttgtataagaaacagaccttgatttccttctgtaaaatcatgcatgtgttcgttttgaattggagtcggcgcgcctgtgttttgaccgtcaggaaagtcttttttttccctgaatagtcaagggtctatacttcttgaagcaattgggacactaatcaattattgtttatacctcggaccatcttttccttcttcacaccactaatcagtttatgccttggaccattaattgtgttgttcacaagcttcttgtttatggtttacaaagcattcgcctagatttgtgtgtgtctctacacatcgatcacttttaaatacttgtcgctttcagttattcttttaacgtttggttatttatcttatttaaaaaaattatcgtattattatttattttgtttgtgatttactttattatcaaaagtatttcaaatatgacttatctttttttataagtgcactaatttttcaaataagatgaatggtcaaatgttacaagaaaaagttaaagcaaccactaatttagggcggaggtagtaaaacctagttattgtaaccaataattttatcaatctataaatgcaacacaaagtaacttcgtgatatctaacacaaagccacttcaacgatgaaagctgactgcatgttttatcaaaacacatgtgatcagtttgttggatgaaaaaaattatctatgtcataaatcaagagttataatataagcttctggctctacaagtaacatttctatgtttttttttacgttcttacatactatgttttgccaaaaaaaacatgatcattttgttggacgaaaagaaatagtaaatatagagtgacctttgatatcattataatataagcttctgcctctataaataacatctatgcactttttacgtcgtagtaatttgatatatgagaaatttacatataacatttttgtgcagcataaccaccatggatttgagcaatagctcacctgtcatcaccgatccggtggcgatcagccagcagttgttgggcggcctgccttcaaatctgatgcagttttcagtcatgcccggtggctactccagctctggcatgaacgttggtgtcagtaggctcaaaatcgaggaagtccttgtcaatggactgcttgatgccatgaaatcctcgtcacctcgcaggaggctgaatgtagcatttggcgaggacaattcatctgaagaagaagaccctgcttacagcgcttggatggcaaaatgtccttctgctttggcttccttcaagcaaattgtagccagtgcacaagggaagaagattgctgtgtttctagactatgacggcacactgtcgcctattgtggatgatcctgacaaagcagtgatgtctcccgtgatgagagctgctgtgagaaatgttgcgaagtacttccccactgcaattgtcagcggaaggtcccgcaataaggtgtttgaatttgtaaaactgaaggagctttattatgctggaagtcatggtatggacataatggcaccttcagcaaatcatgagcacagtgctgaaaagagcaaacaggccaatctcttccaacctgcacacgactttctgccaatgatcgatgaggttaccaagtccctcttgcaagttgtcagtggaattgaaggtgcaactgttgagaacaacaaattctgcgtttctgtagattatcgcaacgttgcagagaaggattggaaactggtcgcacggctcgtaaacgaagtgctggaggcttttcctcgtctcaaagtaaccaatggacgaatggttttagaggttcgtccggtgatcgactgggacaagggaaaggctgtggagtttctgctccagtcactcgggctaaatgactctgaaaatgtgatccccatctacattggagacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtgagctcgctaagcagccatcgatcagctgtcagaagttggagctaataataaaagggatgtggagtgggctacatgtatctcggatctctctgcgagccacctaatggtcttgcgtggccctttaatctgtatgtttttgtgtgtaagctactgctagctgtttgcaccttctgcgtccgtggttgtgtttccgtgctacctttttatgttttgatttggatcttgtttgaaaataatcttaccagctttgggtaaactgtttattacgtactctatatagcatatgtgaccgacgacaacggtttcattttagatgatgtgtatggatgatttctttccaaaatcacatctttagtataagagcaattttaccatccaataccaaattttatactagaaaatattttgggatatcaaaatttatggtacctccagtaccaaatgttgaatggtaaactttcataatatacaagtcactctaggatatttaagacaatttttagttttttcttattgttgcccttgttaaatacatgagaaattttacatcacttaaaatgtatcaagaggtatcaaatttttttaatacaaaatttagtactttctccgtttatatatgaatgtggacaatgcttgaaagtcttataacctgaaactgaggtagtgtatcgagaagtacaaaattttacactaaaatcccagtacttactcaataactgtaaaattactctaaatatgtactccctctatttcagattataagtcgttttaactttagtcaaagttaaactgtttcaagtttaaccaagtttgtagataaaagtagtaacatattcaacacaagacaaatatattataaaaacatattgaattatagatttaattaaattaatttggtattgcaagtattactaaatttgtttataaatttggtcgaatttaaaatagtttgactttaaccaaagtcaaaacaaattataatctaaaacaaaggtaatacattgtatcactctcatgaatggattgtaacatacattaatttaattactattttagttcttgtgcaaaagttgaaaacgattt

SEQ ID NO: 28 - DNA, 4407 bp, Zea mays
agacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtgagctcgctaagcagccatcgatcagctgtcagaagttggagctaataataaaagggatgtggagtgggctacatgtatctcggatctctctgcgagccacctaatggtcttgcgtggccctttaatctgtatgtttttgtgtgtaagctactgctagctgtttgcaccttctgcgtccgtggttgtgtttccgtgctacctttttatgttttgatttggatcttgtttgaaaataatcttaccagctttgggtaaactgtttattacgtactctatatagcatatgtgaccgacgacaacggtttcattttagatgatgtgtatggatgatttctttccaaaatcacatctttagtataagagcaattttaccatccaataccaaattttatactagaaaatattttgggatatcaaaatttatggtacctccagtaccaaatgttgaatggtaaactttcataatatacaagtcactctaggatatttaagacaatttttagttttttcttattgttgcccttgttaaatacatgagaaattttacatcacttaaaatgtatcaagaggtatcaaatttttttaatacaaaatttagtactttctccgtttatatatgaatgtggacaatgcttgaaagtcttataacctgaaactgaggtagtgtatcgagaagtacaaaattttacactaaaatcccagtacttactcaataactgtaaaattactctaaatatgtactccctctatttcagattataagtcgttttaactttagtcaaagttaaactgtttcaagtttaaccaagtttgtagataaaagtagtaacatattcaacacaagacaaatatattataaaaacatattgaattatagatttaattaaattaatttggtattgcaagtattactaaatttgtttataaatttggtcgaatttaaaatagtttgactttaaccaaagtcaaaacaaattataatctaaaacaaaggtaatacattgtatcactctcatgaatggattgtaacatacattaatttaattactattttagttcttgtgcaaaagttgaaaacgatttatgtttggaatctttttgtggtgtatatatatgaaaccattcctctaccatccttccccaaccataatcctcacaaccgttagccccattgtgatctcacccagttgctagcctcttttgtcaccttgtcacagctctcctccattcattacacaatggcatcggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgaactagtgatatcggcgccatgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagtcggggaggggagcgtgagttgtagtcgcggtgtgatc

SEQ ID NO: 29 - DNA, 1116 bp, Oryza sativa
atggatttgagcaatagctcacctgtcatcaccgatccggtggcgatcagccagcagttgttgggcggcctgccttcaaatctgatgcagttttcagtcatgcccggtggctactccagctctggcatgaacgttggtgtcagtaggctcaaaatcgaggaagtccttgtcaatggactgcttgatgccatgaaatcctcgtcacctcgcaggaggctgaatgtagcatttggcgaggacaattcatctgaagaagaagaccctgcttacagcgcttggatggcaaaatgtccttctgctttggcttccttcaagcaaattgtagccagtgcacaagggaagaagattgctgtgtttctagactatgacggcacactgtcgcctattgtggatgatcctgacaaagcagtgatgtctcccgtgatgagagctgctgtgagaaatgttgcgaagtacttccccactgcaattgtcagcggaaggtcccgcaataaggtgtttgaatttgtaaaactgaaggagctttattatgctggaagtcatggtatggacataatggcaccttcagcaaatcatgagcacagtgctgaaaagagcaaacaggccaatctcttccaacctgcacacgactttctgccaatgatcgatgaggttaccaagtccctcttgcaagttgtcagtggaattgaaggtgcaactgttgagaacaacaaattctgcgtttctgtacattatcgcaacgttgcagagaaggattggaaactggtcgcacggctcgtaaacgaagtgctggaggcttttcctcgtctcaaagtaaccaatggacgaatggttttagaggttcgtccggtgatcgactgggacaagggaaaggctgtggagtttctgctccagtcactcgggctaaatgactctgaaaatgtgatccccatctacattggagacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtga

SEQ ID NO: 30 - DNA, 15810 bp, Artificial Sequence
  binary construct 15777
aattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggaccgctaggacgatggtgtgatgtgggaacacgaagaaaacatgaggaaaaaatattaaaatgaatttcccacttaaaatgcatcaaataaaaaaaataaagaaacgaccgggaatagacacagggtttgtgaactagctagggcaaacatcatatggtcccttgctgatgcacaagtacattgagatgtcatttcaattctgtgcatcatatgcatgtggtcccttgctgaatattactcttgaaatatctaccagtgccaatctattgcatgacttaattaattcacaggttttgttgattacattattagtaagcttgagagcacaagctcaatggatttttctataaatggggatcattttgcaattttctttgtcgtgcaaagttagccttctttattactacttctgtttttaaatatacgatcctattgacttttggtcatatatttaaccatgtatcttatttagatagtttgcgcaaatatatataccttcaatgataaaattagttacaatgaaacaaatgatatttacgcaattctttttactaaacaagtcacaagaagtacctgcagcaatatatgttggaaccgtgcagtagatcgagcctagctacgcaaaaaaacaaaaagagaaaaaaagggaaaggaaaaacattaatcatgcatgagcagtatgcccggcaactggaatttgtcaaagatatggggagaggagaataatacaagtactactactacctagctctaccatgcatatgcacccaaaggcaaactggattattggataaagcacagatgctggcaaaacaatccttaagcctcccctccctgcttctttatttttgggcagcctctaccggacggtgccgtggtccattggaccagtaggtggcgacatacatggtttgggttaagtctaggagagcagtgtgtgtgcgcgcgcaagagagagagactgtgagtctgggagtagccctctcccctcctttggccatcttcctcgtgtatatgcatatatgcatcatcgcaacggtgtatatttgtggtgtggcgggtgtggcattggattgcccccattttggctcgtgcttcccagttagggtaaaacctgtggtaaacttgctagccccacgccaaagttacccttctttattgttgaaagggagaggaggtgtgtgaattgtgatggagggagagagagagagatagaaagagagatgtgtgtcaaagcaagcaagaaaccagtttcacaaagagctactactagtactagtgtactactgtggtacagtgcccaatgtcctttctccggactcgactccactaatattctcctcttctcgcgcggctcgttatattctcgtcatcattggaggctttagcaagcaagaagagaggcagtggtggtggtggtggaggaggagctagctagcctgtgcttgctgatcggtgctgagctgaggaatcgttcgatcgatcgggcgagtcgacgaggggaagagttgagctgaggcgcatcgagaacaagatcaacaggcaggtcaccttctccaagcgccgcaacggcctcctcaagaaggcctacgagctgtccgttctctgcgacgccgaggtcgcgctcatcatcttctccagccgcggcaagctctacgagttcggcagcgccgggtataattaatacagacacaacaacacacacaaccaacaaaccagcatcaatttgaacctgcagatctgctgttttctctgatcaattgcttctttttttttgttcttttttgtttcttttatctgctgcaacggcgtcctgctcctctggggtttctcgttttctttttcatttatttttagcaggtgccaagtagccgagctactatacttacctggccatgttaattattttattccgtctgtctgtgtgtgtctgtgcatactactatagggacatggcgcggtgttcttataaaccgggaggccggatccctaactagcatgggaggatatcttttcagcggatctatacaaaccctactcctgctgacctctttcttccagtttctccgggtcttccttggattattattgcccatcttccgggttgtgcgtgtgtcagagacagctcgaacgataaatttctcaaaaccagtactagagagggtgtgttgtgtgtgagaactgagtggagagttagcatgaaggctgcaaactagaaaggaaggtatgttctttcctttttgatccatcaggggagccccttctggtattaagatctttccggcacattgattttcatactttgtgatgaccctggaagaatcggcgtagcagcgtagcaccgctccattttggtcttaccctcacctccccatgctatgaactgatcaatttcattgttcttcatcacccttctcctagctttccacttccttcggatctcatgccatgtttctcagcatgaatcaaatttaattcgtgttttctacttccatatatactggaagaaatttaattagatctatttttgctcgggaggtcttcatactttgagttctgatgccatcaccttatttcccccccccccttctcttgttctatcttcttcctcatcttggcttgatcattttgatctgtcagttatagcatgatgcattctcaatttgactgtatgtaagttcaaccggaaatatgttgaatggattttctatatatcaacacttgatgtcaggcctgcatctgtttcgcttgtggtggtgtggccaaaattgtctatatttgatctttgctcttctttctcctcatttcatgacgattcctactacggcttaaaccattctttattctttactaatcatggatgttgcttgactcctagttgtttcgtactagctcaacttggagatcttttcattatttgcctagttggtgggtacgtttgtgacagatctaaaatggtgcacgaaaagttttacttattatgaaaaaagggagcttaacagggtaatttctctatttattcgtgatgacattttttccttgataagggggattttttataatctgcactcacatgtttatatgtaaaatctagctcttttgttttgtttttggcatatttcccgctaagtatagagtttatgtggataacattataacttttcaagatccaatccacatctttgattgtgaaaatcatacaatagggaaaatcaactgaagggttaattagatgctatatgcatatatatatatatgtgcgcgcgcgcgcgcctgaatttaactatgtatgcatccaactgtttcattgaaaaagatttgatatttttcagtctattctttttcgagtatatatttaatatgtttcaatctgttttgaccattataagataaagcctatattcaccaggcatttgagatgatcttttcatgcatgaaaaagctgttgttatcacttcaactaaccagacgatctaacatgtatttgtataagaaacagaccttgatttccttctgtaaaatcatgcatgtgttcgttttgaattggagtcggcgcgcctgtgttttgaccgtcaggaaagtcttttttttccctgaatagtcaagggtctatacttcttgaagcaattgggacactaatcaattattgtttatacctcggaccatcttttccttcttcacaccactaatcagtttatgccttggaccattaattgtgttgttcacaagcttcttgtttatggtttacaaagcattcgcctagatttgtgtgtgtctctacacatcgatcacttttaaatacttgtcgctttcagttattcttttaacgtttggttatttatcttatttaaaaaaattatcgtattattatttattttgtttgtgatttactttattatcaaaagtatttcaaatatgacttatctttttttataagtgcactaatttttcaaataagatgaatggtcaaatgttacaagaaaaagttaaagcaaccactaatttagggcggaggtagtaaaacctagttattgtaaccaataattttatcaatctataaatgcaacacaaagtcacttcgtgatatctcacacaaagccacttcaacgatgaaagctgactgcatgttttatcaaaacacatgtgatcagtttgttggatgaaaaaaattatctatgtcataaatcaagagttataatataagcttctggctctacaagtaacatttctatgtttttttttacgttcttacatactatgttttgccaaaaaaaacatgatcattttgttggacgaaaagaaatagtaaatatagagtgacctttgatatcattataatataagcttctgcctctataaataacatctatgcactttttacgtcgtagtaatttgatatatgagaaatttacatataacatttttgtgcagcataaccaccatggatttgagcaatagctcacctgtcatcaccgatccggtggcgatcagccagcagttgttgggcggcctgccttcaaatctgatgcagttttcagtcatgcccggtggctactccagctctggcatgaacgttggtgtcagtaggctcaaaatcgaggaagtccttgtcaatggactgcttgatgccatgaaatcctcgtcacctcgcaggaggctgaatgtagcatttggcgaggacaattcatctgaagaagaagaccctgcttacagcgcttggatggcaaaatgtccttctgctttggcttccttcaagcaaattgtagccagtgcacaagggaagaagattgctgtgtttctagactatgacggcacactgtcgcctattgtggatgatcctgacaaagcagtgatgtctcccgtgatgagagctgctgtgagaaatgttgcgaagtacttccccactgcaattgtcagcggaaggtcccgcaataaggtgtttgaatttgtaaaactgaaggagctttattatgctggaagtcatggtatggacataatggcaccttcagcaaatcatgagcacagtgctgaaaagagcaaacaggccaatctcttccaacctgcacacgactttctgccaatgatcgatgaggttaccaagtccctcttgcaagttgtcagtggaattgaaggtgcaactgttgagaacaacaaattctgcgtttctgtacattatcgcaacgttgcagagaaggattggaaactggtcgcacggctcgtaaacgaagtgctggaggcttttcctcgtctcaaagtaaccaatggacgaatggttttagaggttcgtccggtgatcgactgggacaagggaaaggctgtggagtttctgctccagtcactcgggctaaatgactctgaaaatgtgatccccatctacattggagacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtgagctcgctaagcagccatcgatcagctgtcagaagttggagctaataataaaagggatgtggagtgggctacatgtatctcggatctctctgcgagccacctaatggtcttgcgtggccctttaatctgtatgtttttgtgtgtaagctactgctagctgtttgcaccttctgcgtccgtggttgtgtttccgtgctacctttttatgttttgatttggatcttgtttgaaaataatcttaccagctttgggtaaactgtttattacgtactctatatagcatatgtgaccgacgacaacggtttcattttagatgatgtgtatggatgatttctttccaaaatcacatctttagtataagagcaattttaccatccaataccaaattttatactagaaaatattttgggatatcaaaatttatggtacctccagtaccaaatgttgaatggtaaactttcataatatacaagtcactctaggatatttaagacaatttttagttttttcttattgttgcccttgttaaatacatgagaaattttacatcacttaaaatgtatcaagaggtatcaaatttttttaatacaaaatttagtactttctccgtttatatatgaatgtggacaatgcttgaaagtcttataacctgaaactgaggtagtgtatcgagaagtacaaaattttacactaaaatcccagtacttactcaataactgtaaaattactctaaatatgtactccctctatttcagattataagtcgttttaactttagtcaaagttaaactgtttcaagtttaaccaagtttgtagataaaagtagtaacatattcaacacaagacaaatatattataaaaacatattgaattatagatttaattaaattaatttggtattgcaagtattactaaatttgtttataaatttggtcgaatttaaaatagtttgactttaaccaaagtcaaaacaaattataatctaaaacaaaggtaatacattgtatcactctcatgaatggattgtaacatacattaatttaattactattttagttcttgtgcaaaagttgaaaacgatttatgtttggaatctttttgtggtgtatatatatgaaaccattcctctaccatccttccccaaccataatcctcacaaccgttagccccattgtgatctcacccagttgctagcctcttttgtcaccttgtcacagctctcctccattcattacacaatggcatcggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgaactagtgatatcggcgccatgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatt

SEQ ID NO: 31 - DNA, 15329 bp, Artificial Sequence
  binary construct 15769
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtaccggaccgctaggacgatggtgtgatgtgggaacacgaagaaaacatgaggaaaaaatattaaaatgaatttcccacttaaaatgcatcaaataaaaaaaataaagaaacgaccgggaatagacacagggtttgtgaactagctagggcaaacatcatatggtcccttgctgatgcacaagtacattgagatgtcatttcaattctgtgcatcatatgcatgtggtcccttgctgaatattactcttgaaatatctaccagtgccaatctattgcatgacttaattaattcacaggttttgttgattacattattagtaagcttgagagcacaagctcaatggatttttctataaatggggatcattttgcaattttctttgtcgtgcaaagttagccttctttattactacttctgtttttaaatatacgatcctattgacttttggtcatatatttaaccatgtatcttatttagatagtttgcgcaaatatatataccttcaatgataaaattagttacaatgaaacaaatgatatttacgcaattctttttactaaacaagtcacaagaagtacctgcagcaatatatgttggaaccgtgcagtagatcgagcctagctacgcaaaaaaacaaaaagagaaaaaaagggaaaggaaaaacattaatcatgcatgagcagtatgcccggcaactggaatttgtcaaagatatggggagaggagaataatacaagtactactactacctagctctaccatgcatatgcacccaaaggcaaactggattattggataaagcacagatgctggcaaaacaatccttaagcctcccctccctgcttctttatttttgggcagcctctaccggacggtgccgtggtccattggaccagtaggtggcgacatacatggtttgggttaagtctaggagagcagtgtgtgtgcgcgcgcaagagagagagactgtgagtctgggagtagccctctcccctcctttggccatcttcctcgtgtatatgcatatatgcatcatcgcaacggtgtatatttgtggtgtggcgggtgtggcattggattgcccccattttggctcgtgcttcccagttagggtaaaacctgtggtaaacttgctagccccacgccaaagttacccttctttattgttgaaagggagaggaggtgtgtgaattgtgatggagggagagagagagagatagaaagagagatgtgtgtcaaagcaagcaagaaaccagtttcacaaagagctactactagtactagtgtactactgtggtacagtgcccaatgtcctttctccggactcgactccactaatattctcctcttctcgcgcggctcgttatattctcgtcatcattggaggctttagcaagcaagaagagaggcagtggtggtggtggtggaggaggagctagctagcctgtgcttgctgatcggtgctgagctgaggaatcgttcgatcgatcgggcgagtcgacgaggggaagagttgagctgaggcgcatcgagaacaagatcaacaggcaggtcaccttctccaagcgccgcaacggcctcctcaagaaggcctacgagctgtccgttctctgcgacgccgaggtcgcgctcatcatcttctccagccgcggcaagctctacgagttcggcagcgccgggtataattaatacagacacaacaacacacacaaccaacaaaccagcatcaatttgaacctgcagatctgctgttttctctgatcaattgcttctttttttttgttcttttttgtttcttttatctgctgcaacggcgtcctgctcctctggggtttctcgttttctttttcatttatttttagcaggtgccaagtagccgagctactatacttacctggccatgttaattattttattccgtctgtctgtgtgtgtctgtgcatactactatagggacatggcgcggtgttcttataaaccgggaggccggatccctaactagcatgggaggatatcttttcagcggatctatacaaaccctactcctgctgacctctttcttccagtttctccgggtcttccttggattattattgcccatcttccgggttgtgcgtgtgtcagagacagctcgaacgataaatttctcaaaaccagtactagagagggtgtgttgtgtgtgagaactgagtggagagttagcatgaaggctgcaaactagaaaggaaggtatgttctttcctttttgatccatcaggggagccccttctggtattaagatctttccggcacattgattttcatactttgtgatgaccctggaagaatcggcgtagcagcgtagcaccgctccattttggtcttaccctcacctccccatgctatgaactgatcaatttcattgttcttcatcacccttctcctagctttccacttccttcggatctcatgccatgtttctcagcatgaatcaaatttaattcgtgttttctacttccatatatactggaagaaatttaattagatctatttttgctcgggaggtcttcatactttgagttctgatgccatcaccttatttcccccccccccttctcttgttctatcttcttcctcatcttggcttgatcattttgatctgtcagttatagcatgatgcattctcaatttgactgtatgtaagttcaaccggaaatatgttgaatggattttctatatatcaacacttgatgtcaggcctgcatctgtttcgcttgtggtggtgtggccaaaattgtctatatttgatctttgctcttctttctcctcatttcatgacgattcctactacggcttaaaccattctttattctttactaatcatggatgttgcttgactcctagttgtttcgtactagctcaacttggagatcttttcattatttgcctagttggtgggtacgtttgtgacagatctaaaatggtgcacgaaaagttttacttattatgaaaaaagggagcttaacagggtaatttctctatttattcgtgatgacattttttccttgataagggggattttttataatctgcactcacatgtttatatgtaaaatctagctcttttgttttgtttttggcatatttcccgctaagtatagagtttatgtggataacattataacttttcaagatccaatccacatctttgattgtgaaaatcatacaatagggaaaatcaactgaagggttaattagatgctatatgcatatatatatatatgtgcgcgcgcgcgcgcctgaatttaactatgtatgcatccaactgtttcattgaaaaagatttgatatttttcagtctattctttttcgagtatatatttaatatgtttcaatctgttttgaccattataagataaagcctatattcaccaggcatttgagatgatcttttcatgcatgaaaaagctgttgttatcacttcaactaaccagacgatctaacatgtatttgtataagaaacagaccttgatttccttctgtaaaatcatgcatgtgttcgttttgaattggagtcggcgcgcctgtgttttgaccgtcaggaaagtcttttttttccctgaatagtcaagggtctatacttcttgaagcaattgggacactaatcaattattgtttatacctcggaccatcttttccttcttcacaccactaatcagtttatgccttggaccattaattgtgttgttcacaagcttcttgtttatggtttacaaagcattcgcctagatttgtgtgtgtctctacacatcgatcacttttaaatacttgtcgctttcagttattcttttaacgtttggttatttatcttatttaaaaaaattatcgtattattatttattttgtttgtgatttactttattatcaaaagtatttcaaatatgacttatctttttttataagtgcactaatttttcaaataagatgaatggtcaaatgttacaagaaaaagttaaagcaaccactaatttagggcggaggtagtaaaacctagttattgtaaccaataattttatcaatctataaatgcaacacaaagtcacttcgtgatatctcacacaaagccacttcaacgatgaaagctgactgcatgttttatcaaaacacatgtgatcagtttgttggatgaaaaaaattatctatgtcataaatcaagagttataatataagcttctggctctacaagtaacatttctatgtttttttttacgttcttacatactatgttttgccaaaaaaaacatgatcattttgttggacgaaaagaaatagtaaatatagagtgacctttgatatcattataatataagcttctgcctctataaataacatctatgcactttttacgtcgtagtaatttgatatatgagaaatttacatataacatttttgtgcagcataaccaccatggatttgagcaatagctcacctgtcatcaccgatccggtggcgatcagccagcagttgttgggcggcctgccttcaaatctgatgcagttttcagtcatgcccggtggctactccagctctggcatgaacgttggtgtcagtaggctcaaaatcgaggaagtccttgtcaatggactgcttgatgccatgaaatcctcgtcacctcgcaggaggctgaatgtagcatttggcgaggacaattcatctgaagaagaagaccctgcttacagcgcttggatggcaaaatgtccttctgctttggcttccttcaagcaaattgtagccagtgcacaagggaagaagattgctgtgtttctagactatgacggcacactgtcgcctattgtggatgatcctgacaaagcagtgatgtctcccgtgatgagagctgctgtgagaaatgttgcgaagtacttccccactgcaattgtcagcggaaggtcccgcaataaggtgtttgaatttgtaaaactgaaggagctttattatgctggaagtcatggtatggacataatggcaccttcagcaaatcatgagcacagtgctgaaaagagcaaacaggccaatctcttccaacctgcacacgactttctgccaatgatcgatgaggttaccaagtccctcttgcaagttgtcagtggaattgaaggtgcaactgttgagaacaacaaattctgcgtttctgtacattatcgcaacgttgcagagaaggattggaaactggtcgcacggctcgtaaacgaagtgctggaggcttttcctcgtctcaaagtaaccaatggacgaatggttttagaggttcgtccggtgatcgactgggacaagggaaaggctgtggagtttctgctccagtcactcgggctaaatgactctgaaaatgtgatccccatctacattggagacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtgagctcgctaagcagccatcgatcagctgtcagaagttggagctaataataaaagggatgtggagtgggctacatgtatctcggatctctctgcgagccacctaatggtcttgcgtggccctttaatctgtatgtttttgtgtgtaagctactgctagctgtttgcaccttctgcgtccgtggttgtgtttccgtgctacctttttatgttttgatttggatcttgtttgaaaataatcttaccagctttgggtaaactgtttattacgtactctatatagcatatgtgaccgacgacaacggtttcattttagatgatgtgtatggatgatttctttccaaaatcacatctttagtataagagcaattttaccatccaataccaaattttatactagaaaatattttgggatatcaaaatttatggtacctccagtaccaaatgttgaatggtaaactttcataatatacaagtcactctaggatatttaagacaatttttagttttttcttattgttgcccttgttaaatacatgagaaattttacatcacttaaaatgtatcaagaggtatcaaatttttttaatacaaaatttagtactttctccgtttatatatgaatgtggacaatgcttgaaagtcttataacctgaaactgaggtagtgtatcgagaagtacaaaattttacactaaaatcccagtacttactcaataactgtaaaattactctaaatatgtactccctctatttcagattataagtcgttttaactttagtcaaagttaaactgtttcaagtttaaccaagtttgtagataaaagtagtaacatattcaacacaagacaaatatattataaaaacatattgaattatagatttaattaaattaatttggtattgcaagtattactaaatttgtttataaatttggtcgaatttaaaatagtttgactttaaccaaagtcaaaacaaattataatctaaaacaaaggtaatacattgtatcactctcatgaatggattgtaacatacattaatttaattactattttagttcttgtgcaaaagttgaaaacgatttatgtttggaatctttttgtggtgtatatatatgaaaccattcctctaccatccttccccaaccataatcctcacaaccgttagccccattgtgatctcacccagttgctagcctcttttgtcaccttgtcacagctctcctccattcattacacaatggcatcggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgaactagtgatatcggcgccatgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 32 - DNA, 19 bp, Artificial Sequence
  TaqMan forward primer P23198
gcgtgagttggagtcgtgg

SEQ ID NO: 33 - DNA, 23 bp, Artificial Sequence
  TaqMan reverse primer P23352
attctccgctcatgatcagattg

SEQ ID NO: 34 - DNA, 31 bp, Artificial Sequence
  TaqMan probe labeled with FAM on 5′-terminus and labeled with BHQ1 on 3′-terminus
cactgatagtttaaactgaaggcgggaaacg

SEQ ID NO: 35 - DNA, 80 bp, Artificial Sequence
  TaqMan amplicon
gcgtgagttggagtcgtggaggaaacactgatagtttaaactgaaggcgggaaacgacaatctgatcatgagcggagaat

SEQ ID NO: 36 - DNA, 21 bp, Zea mays
  misc_feature (1)..(19): for detection of 5′ junction
tccaaacgtaaaacggcttgt

SEQ ID NO: 37 - DNA, 139 bp, Zea mays
  misc_feature (1)..(139): amplicon
gcgtgagttggagtcgtggaggaaacactgatagtttaaactgaaggcgggaaacgacaatctgatcatgagcggagaattaagggagtcacgttatgacccccgccgatgacgcgggacaagccgttttacgtttgga

(19) United States
(12) Patent Application Publication
(10) Pub. No.: US 2013/0217019 A1
(43) Pub. Date: Aug. 22, 2013

(54) CORN EVENT MZDT09Y

(71) Applicant: SYNGENTA PARTICIPATIONS AG, (US)

(72) Inventors: Ryan William Carlin, Durham, NC (US); Chunyang Fan, Durham, NC (US); Yanshan Ji, Durham, NC (US)

(73) Assignee: SYNGENTA PARTICIPATIONS AG, Basel (CH)

(21) Appl. No.: 13/873,611

(22) Filed: Apr. 30, 2013

Related U.S. Application Data
(63) Continuation-in-part of application No. 13/549,693, filed on Jul. 16, 2012.
(60) Provisional application No. 61/508,605, filed on Jul. 15, 2011.
(60) Provisional application No. 61/522,549, filed on Aug. 11, 2011.

(51) Int. Cl. C12Q 1/68

(52) U.S. Cl. CPC C12Q 1/6895
USPC 435/6.11; 435/6.12; 536/24.3; 536/24.33

(57) ABSTRACT

A novel transgenic corn event designated MZDT09Y is disclosed. The invention relates to nucleic acids that are unique to event MZDT09Y and to methods of detecting the presence of event MZDT09Y based on DNA sequences of the recombinant constructs inserted into the corn genome that resulted in the MZDT09Y event and of genomic sequences flanking the insertion site. The invention further relates to corn plants comprising the transgenic genotype of event MZDT09Y and to methods for producing a corn plant by crossing a corn plant comprising the MZDT09Y genotype with itself or another corn variety. Seeds of corn plants comprising the MZDT09Y genotype are also objects of the invention.

### RELATED APPLICATIONS

This application is a continuation-in-part of U.S. Non-Provisional application Ser. No. 13/549,693, filed Jul. 16, 2012, which claims the benefit of U.S. Provisional Application No. 61/508,605, filed Jul. 15, 2011, and U.S. Provisional Application No. 61/522,549, filed Aug. 11, 2011. The entire teachings of the above applications are incorporated herein by reference.

### BACKGROUND OF THE INVENTION

The increasing world population and the dwindling supply of arable land available for agriculture fuels the need for research in the area of increasing the efficiency of agriculture. Conventional means for crop and horticultural improvements utilize selective breeding techniques to identify plants having desirable characteristics. However, such selective breeding techniques have several drawbacks, namely that these techniques are often labor intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant's genome. Such technology has the capacity to deliver crops or plants having various improved economic, agronomic or horticultural traits.

### SUMMARY OF THE INVENTION

The following Summary lists several embodiments of the invention subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the invention, whether listed in this Summary or not. To avoid excessive repetition, this Summary does not list or suggest all possible combinations of such features.

Unless otherwise defined, all technical and scientific terms used herein have the same meaning as commonly understood by one of ordinary skill in the art to which this invention belongs.

The invention provides nucleotide sequences that when transgenically expressed in a plant increases plant vigor, yield and/or biomass as well as increased stress tolerance. It was discovered that the T6PP proteins described herein comprise modifications which are significantly associated with increased yield and/or increased tolerance to stress when transgenically expressed in a plant.

In one embodiment, the present invention encompasses a nucleic acid molecule, preferably isolated, comprising a nucleotide sequence that is unique to corn event MZDT09Y. The nucleotide sequence may comprise any one of SEQ ID NO: 1, SEQ ID NO: 2, SEQ ID NO: 3, SEQ ID NO: 4, or any of the complements thereof. The nucleic acid molecule is comprised in a corn seed deposited at the American Type Culture Collection under the accession number PTA-13025.

In another embodiment, the present invention encompasses a pair of polynucleotide primers comprising a first polynucleotide primer and a second polynucleotide primer which function together in the presence of an event MZDT09Y DNA template in a sample to produce an amplicon diagnostic for event MZDT09Y. In one aspect, the pair of the first polynucleotide primer or a sequence of the second polynucleotide primer is chosen from SEQ ID NO: 7, or the complement thereof; or a sequence of the first polynucleotide primer is or is complementary to a corn plant genome sequence flanking the point of insertion of a heterologous DNA sequence inserted into the corn plant genome of event MZDT09Y, and a sequence of the second polynucleotide primer is or is complementary to the heterologous DNA sequence inserted into the genome of event MZDT09Y. In another aspect, the first polynucleotide primer comprises at least 10 contiguous nucleotides of a nucleotide sequence selected from the group consisting of SEQ ID NO: 5, SEQ ID NO: 6, and the complements thereof; and the second polynucleotide primer comprises at least 10 contiguous nucleotides from SEQ ID NO: 7, or the complements thereof. In another aspect, the first polynucleotide primer comprises a nucleotide sequence selected from the group consisting of SEQ ID NO: 12, SEQ ID NO: 14, SEQ ID NO: 32, and the complements thereof; and the second polynucleotide primer comprises a nucleotide sequence selected from the group consisting of SEQ ID NO: 13, SEQ ID NO: 15, SEQ ID NO: 33, and the complements thereof. In another aspect, the first polynucleotide primer consists of SEQ ID NO: 12 and the second polynucleotide primer consists of SEQ ID NO: 13; or the first polynucleotide primer consists of SEQ ID NO: 14 and the second polynucleotide primer consists of SEQ ID NO: 15; or the first polynucleotide primer consists of SEQ ID NO: 32 and the second polynucleotide primer consists of SEQ ID NO: 33.

In another embodiment, the present invention encompasses a method of detecting the presence of a nucleic acid molecule that is unique to event MZDT09Y in a sample comprising corn nucleic acids. In one aspect, the method comprises contacting the sample with a pair of primers that, when used in a nucleic-acid amplification reaction with genomic DNA from event MZDT09Y produces an amplicon that is diagnostic for event MZDT09Y; performing a nucleic acid amplification reaction, thereby producing the amplicon; and detecting the amplicon. In another aspect, the method comprises contacting the sample with a probe that hybridizes under high stringency conditions with genomic DNA from event MZDT09Y and does not hybridize under high stringency conditions with DNA of a control corn plant; subjecting the sample and probe to high stringency hybridization conditions; and detecting hybridization of the probe to the nucleic acid molecule.

In another embodiment, the present invention encompasses a kit for detecting nucleic acids that are unique to event MZDT09Y comprising at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to event MZDT09Y in the sample. In one aspect, the nucleic acid molecule of sufficient length of continuous polynucleotides comprises a nucleotide sequence selected from the group consisting SEQ ID NO: 1, SEQ ID NO: 2, SEQ ID NO: 3, SEQ ID NO: 4, SEQ ID NO: 8; and the complements thereof. In another aspect, the nucleic acid molecule is selected from the group consisting of SEQ ID NOs: 12-15, SEQ ID NOs: 32-34, and the complements thereof.

In another embodiment, the present invention encompasses a transgenic corn plant, or cells or tissues thereof, comprising a nucleic acid molecule that is unique to corn event MZDT09Y. The nucleotide sequence may comprise any one of SEQ ID NO: 1, SEQ ID NO: 2, SEQ ID NO: 3, SEQ ID NO: 4, or any of the complements thereof. In another embodiment, the present invention encompasses a corn seed comprising a nucleic acid molecule that is unique to corn event MZDT09Y. An example of the seed is deposited at the American Type Culture Collection under the accession number PTA-13025.

In another embodiment, the present invention encompasses a biological sample derived from an event MZDT09Y corn plant, tissue, or seed, wherein the sample comprises a nucleotide sequence which is or is complementary to a nucleotide sequence selected from the group consisting SEQ ID NO: 1, SEQ ID NO: 2, SEQ ID NO: 3, SEQ ID NO: 4, and the complements thereof; and wherein the sequence is detectable in the sample using a nucleic acid amplification or nucleic acid hybridization method. In one aspect, the sample is selected from the group consisting of corn flour, corn meal, corn syrup, corn oil, corn starch, and cereals manufactured in whole or in part to contain corn by-products. In another embodiment, the present invention encompasses an extract derived from the biological sample. In one aspect, the extract is selected from the group consisting of corn flour, corn meal, corn syrup, corn oil, corn starch, and cereals manufactured in whole or in part to contain corn by-products.

In another embodiment, the present invention encompasses a method for producing a corn plant with increased yield, the method comprising sexually crossing a first parent corn plant with a second parent corn plant, wherein said first or second parent corn plant comprises event MZDT09Y DNA, thereby producing a plurality of first generation progeny plants; selecting a first generation progeny plant with increased yield; selfing the first generation progeny plant, thereby producing a plurality of second generation progeny plants; and selecting from the second generation progeny plants, a plant with increased yield; wherein the second generation progeny plants comprise a nucleic acid molecule selected from the group consisting of SEQ ID NO: 1, SEQ ID NO: 2, SEQ ID NO: 3, SEQ ID NO: 4, and the complements thereof. In one aspect, the increased yield is indicated by an increase as compared to a control plant of any one of the following: increased grain yield, increased seed, increased seed weight, increased biomass, increased sugar, increased oil, increased plant vigor, increased yield under non-optimal conditions, increased yield under stress conditions and increased yield under water stress conditions.

In another embodiment, the present invention encompasses a method for producing a corn plant with increased tolerance to abiotic stress, the method comprising sexually crossing a first parent corn plant with a second parent corn plant, wherein said first or second parent corn plant comprises event MZDT09Y DNA, thereby producing a plurality of first generation progeny plants; selecting a first generation progeny plant with increased tolerance to abiotic stress; selfing the first generation progeny plant, thereby producing a plurality of second generation progeny plants; and selecting from the second generation progeny plants, a plant with increase tolerance to abiotic stress; wherein the second generation progeny plants comprise a nucleic acid molecule selected from the group consisting of SEQ ID NO: 1, SEQ ID NO: 2, SEQ ID NO: 3, SEQ ID NO: 4, and the complements thereof. In one aspect, the abiotic stress comprises stress selected from the group consisting of water stress, heat stress or cold stress. In another aspect, the water stress is caused by drought.

In another embodiment, the present invention encompasses a method of producing hybrid corn seeds comprising planting seeds of a first inbred corn line comprising event MZDT09Y and seeds of a second inbred line having a genotype different from the first inbred corn line; cultivating corn plants resulting from the planting until time of flowering; emasculating the flowers of plants of one of the corn inbred lines; sexually crossing the two different inbred lines with each other; harvesting the hybrid seed produced thereby. The hybrid seed produced by this method comprise a nucleic acid molecule selected from the group consisting of SEQ ID NO: 1, SEQ ID NO: 2, SEQ ID NO: 3, SEQ ID NO: 4, and the complements thereof. In one aspect, the plants of the first inbred corn line are the female parents or male parents. In another embodiment, the present invention encompasses hybrid seed produced by the above method of producing hybrid seed.

These and other features, objects and advantages of the present invention will become better understood from the description that follows. In the description, reference is made to the accompanying sequences, which form a part hereof and in which there is shown by way of illustration, not limitation, embodiments of the invention. The description of preferred embodiments is not intended to limit the invention to cover all modifications, equivalents and alternatives. Reference should therefore be made to the embodiments recited herein for interpreting the scope of the invention.

### BRIEF DESCRIPTION OF THE SEQUENCES IN THE SEQUENCE LISTING

SEQ ID NO: 1 is the 20 by 5′-junction sequence.

SEQ ID NO: 2 is the 20 by 3′-junction sequence.

SEQ ID NO: 3 is the 60 by 5′-genome plus insert sequence.

SEQ ID NO: 4 is the 60 by 3′-insert plus genome sequence.

SEQ ID NO: 5 is the 5′-flanking genomic sequence.

SEQ ID NO: 6 is the 3′-flanking genomic sequence.

SEQ ID NO: 7 is the heterologous insert sequence.

SEQ ID NO: 8 is the heterologous insert sequence plus genomic flanking sequences.

SEQ ID NO: 9 is the OsMADS6 promoter.

SEQ ID NO: 10 is the sequence of the modified T6PP gene.

SEQ ID NO: 11 is the sequence of the modified T6PP protein.

SEQ ID NOs: 12-22 are primers useful in the present invention.

SEQ ID NOs: 23-28 are amplicons useful in the present invention.

SEQ ID NO: 29 is the unmodified rice T6PP (OsT6PP) cDNA sequence.

SEQ ID NO: 30 is the binary construct 15777.

SEQ ID NO: 31 is the binary construct 15769.

SEQ ID NOs: 32-34 are TaqMan primers and probe

SEQ ID NO: 35 is the amplicon produced by the TaqMan PCR reaction.

SEQ ID NO: 36 is a primer useful in the present invention

SEQ ID NO: 37 is an amplicon useful in the present invention

### DETAILED DESCRIPTION OF THE INVENTION

The practice of the present invention will employ, unless otherwise indicated, conventional techniques of botany, microbiology, tissue culture, molecular biology, chemistry, biochemistry, plant quantitative genetics, statistics and recombinant DNA technology, which are within the skill of the art. Such techniques are explained fully in the literature. See, e.g., Langenheim and Thimann, (1982) Botany: Plant Biology and Its Relation to Human Affairs, John Wiley; Cell Culture and Somatic Cell Genetics of Plants, vol. 1, Vasil, ed. (1984); Stanier, et al., (1986) The Microbial World, 5th ed., Prentice-Hall; Dhringra and Sinclair, (1985) Basic Plant Pathology Methods, CRC Press; Maniatis, et al., (1982) Molecular Cloning: A Laboratory Manual; DNA Cloning, vols. I and II, Glover, ed. (1985); Oligonucleotide Synthesis, Gait, ed. (1984); Nucleic Acid Hybridization, Hames and Higgins, eds. (1984); and the series Methods in Enzymology, Colowick and Kaplan, eds, Academic Press, Inc., San Diego, Calif.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention.

As used herein the singular forms “a”, “and”, and “the” include plural reference unless the context clearly dictates otherwise. Thus, for example, reference to “a vector” is a reference to one or more vectors and includes equivalents thereof known to those skilled in the art.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent.

As used herein, the word “or” means any one member of a particular list and also includes any combination of members on that list.

The terms “comprises”, “comprising”, “includes”, “including”, “having” and their conjugates mean “including but not limited to”. The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), O-Beta Replicase systems, transcription-based amplification system (TAS) and strand displacement amplification (SDA). See, e.g., Diagnostic Molecular Microbiology: Principles and Applications, Persing, et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present invention, is implicit in each described polypeptide sequence and incorporated herein by reference.

A “control plant” or “control” as used herein may be a non-transgenic plant of the parental line used to generate a transgenic plant herein. A control plant may in some cases be a transgenic plant line that includes an empty vector or marker gene, but does not contain the recombinant polynucleotide of the present invention that is expressed in the transgenic plant being evaluated. A control plant in other cases is a transgenic plant expressing the gene with a constitutive promoter. In general, a control plant is a plant of the same line or variety as the transgenic plant being tested, lacking the specific trait-conferring, recombinant DNA that characterizes the transgenic plant. Such a progenitor plant that lacks that specific trait-conferring recombinant DNA can be a natural, wild-type plant, an elite, non-transgenic plant, or a transgenic plant without the specific trait-conferring, recombinant DNA that characterizes the transgenic plant. The progenitor plant lacking the specific, trait-conferring recombinant DNA can be a sibling of a transgenic plant having the specific, trait-conferring recombinant DNA. Such a progenitor sibling plant may include other recombinant DNA

As to amino acid sequences, one of skill will recognize that individual substitutions, deletions or additions to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity or ligand/receptor binding is generally at least 30%, 40%, 50%, 60%, 70%, 80% or 90%, preferably 60-90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V) and

6) Phenylalanine (F), Tyrosine (Y), Tryptophan (W).

See also, Creighton, Proteins, W.H. Freeman and Co. (1984).

As used herein the terms “modified” or “modification” interchangeably refer to deliberate or random substitutions, deletions or additions to a nucleic acid, peptide, polypeptide or protein sequence which alters, adds or deletes at least one amino acid residue within a given polypeptide. A “modified T6PP” as used herein refers to any nucleic acid encoding a T6PP or peptides, polypeptides or protein having T6PP activity either of which having been modified so that the resultant T6PP confers decreased T6PP activity and/or decreased binding to T6P.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolumn (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, a “heterologous” nucleic acid sequence is a nucleic acid sequence not naturally associated with a host cell into which it is introduced, including non-naturally occurring multiple copies of a naturally occurring nucleic acid sequence. A heterologous nucleic acid refers to a nucleic acid that originates from a foreign species with respect to a host cell or from the same species as the host cell, provided the heterologous nucleic acid sequence is substantially modified from its native form in composition and/or genomic locus by deliberate human intervention. A heterologous protein may originate from a foreign species, or if from the same species, is substantially modified from its original form by deliberate human intervention.

By “host cell” is meant a cell, which comprises a heterologous nucleic acid sequence of the invention, which comprises an expression cassette and supports the replication and/or expression of the expression cassette. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

As used herein, the term transgenic “event” refers to a recombinant plant produced by transformation and regeneration of a plant cell or tissue with heterologous DNA, for example, an expression cassette that includes a gene of interest. The term “event” refers to the original transformant and/or progeny of the transformant that include the heterologous DNA. The term “event” also refers to progeny produced by a sexual outcross between the transformant and another corn line. Even after repeated backcrossing to a recurrent parent, the inserted DNA and the flanking DNA from the transformed parent is present in the progeny of the cross at the same chromosomal location. The term “event” also refers to DNA from the original transformant comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA. Normally, transformation of plant tissue produces multiple events, each of which represent insertion of a DNA construct into a different location in the genome of a plant cell. Based on the expression of the transgene or other desirable characteristics, a particular event is selected. Thus, “event MZDT09Y”, “MZDT09Y”, “09Y” or “09Y event” may be used interchangeably.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

As used herein “gene stack” refers to the introduction of two or more genes into the genome of an organism. In certain aspects of the invention it may be desirable to stack any abiotic stress gene (e.g. cold shock proteins, genes associated with ABA response) with the T6PPs as described herein. Likewise, it may also be desirable to stack the T6PPs as described herein with genes conferring insect resistance, disease resistance, increased yield or any other beneficial trait (e.g. increased plant height, etc.) known in the art.

The terms “isolated” refers to material, such as a nucleic acid or a protein, which is substantially or essentially free from components which normally accompany or interact with it as found in its naturally occurring environment. The isolated material optionally comprises material not found with the material in its natural environment. In contrast, a non-isolated nucleic acid, such as DNA or RNA, is found in the state in which it exists in nature. An isolated nucleic acid may be in a transgenic plant and still be considered “isolated.” Nucleic acids, which are “isolated,” as defined herein, are also referred to as “heterologous” nucleic acids.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise in one case a substantial representation of the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2nd ed., vols. 1-3; and Current Protocols in Molecular Biology, Ausubel, et al., eds, Current Protocols, a joint venture between Greene Publishing Associates, Inc. and John Wiley & Sons, Inc. (1994 Supplement). In another instance “nucleic acid library” as defined herein may also be understood to represent libraries comprising a prescribed faction or rather not substantially representing an entire genome of a specified organism. For example, small RNAs, mRNAs and methylated DNA. A nucleic acid library as defined herein might also encompass variants of a particular molecule (e.g. a collection of variants for a particular protein).

As used herein “operably linked” includes reference to a functional linkage between a first sequence, such as a promoter and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs, tissues (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the invention, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” may include reference to bushels per acre of a grain crop at harvest, as adjusted for grain moisture (15% typically for maize, for example), and the volume of biomass generated (for forage crops such as alfalfa and plant root size for multiple crops). Grain moisture is measured in the grain at harvest. The adjusted test weight of grain is determined to be the weight in pounds per bushel, adjusted for grain moisture level at harvest. Biomass is measured as the weight of harvestable plant material generated. Yield can be affected by many properties including without limitation, plant height, pod number, pod position on the plant, number of internodes, incidence of pod shatter, grain size, efficiency of nodulation and nitrogen fixation, efficiency of nutrient assimilation, carbon assimilation, plant architecture, percent seed germination, seedling vigor, and juvenile traits. Yield can also be affected by efficiency of germination (including germination in stressed conditions), growth rate (including growth rate in stressed conditions), ear number, seed number per ear, seed size, composition of seed (starch, oil, protein) and characteristics of seed fill. Yield of a plant of the can be measured in a number of ways, including test weight, seed number per plant, seed weight, seed number per unit area (i.e. seeds, or weight of seeds, per acre), bushels per acre, tons per acre, or kilo per hectare. For example, corn yield may be measured as production of shelled corn kernels per unit of production area, for example in bushels per acre or metric tons per hectare, often reported on a moisture adjusted basis, for example at 15.5 percent moisture. Moreover a bushel of corn is defined by law in the State of Iowa as 56 pounds by weight, a useful conversion factor for corn yield is: 100 bushels per acre is equivalent to 6.272 metric tons per hectare. Other measurements for yield are common practice in the art In certain embodiments of the invention yield may be increased in stressed and/or non-stressed conditions.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions in most cells.

Any suitable promoter sequence can be used by the nucleic acid construct of the present invention. According to some embodiments of the invention, the promoter is a constitutive promoter, a tissue-specific, or an abiotic stress-inducible promoter.

Suitable constitutive promoters include, for example, CaMV 35S promoter (SEQ ID NO:1546; Odell et al., Nature 313:810-812, 1985); Arabidopsis At6669 promoter (SEQ ID NO:1652; see PCT Publication No. WO04081173A2); maize Ubi 1 (Christensen et al., Plant Mol. Biol. 18:675-689, 1992); rice actin (McElroy et al., Plant Cell 2:163-171, 1990); pEMU (Last et al., Theor. Appl. Genet. 81:581-588, 1991); CaMV 19S (Nilsson et al., Physiol. Plant 100:456-462, 1997); GOS2 (de Pater et al., Plant J November; 2(6):837-44, 1992); ubiquitin (Christensen et al., Plant Mol. Biol. 18: 675-689, 1992); Rice cyclophilin (Bucholz et al., Plant Mol. Biol. 25(5):837-43, 1994); Maize H3 histone (Lepetit et al., Mol. Gen. Genet. 231: 276-285, 1992); Actin 2 (An et al., Plant J. 10(1); 107-121, 1996), constitutive root tip CT2 promoter (SEQ ID NO:1535; see also PCT application No. IL/2005/000627) and Synthetic Super MAS (Ni et al., The Plant Journal 7: 661-76, 1995). Other constitutive promoters include those in U.S. Pat. Nos. 5,659,026, 5,608,149; 5,608,144; 5,604,121; 5,569,597: 5,466,785; 5,399,680; 5,268,463; and 5,608,142.

Suitable tissue-specific promoters include, but not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed specific genes (Simon, et al., Plant Mol. Biol. 5. 191, 1985; Scofield, et al., J. Biol. Chem. 262: 12202, 1987; Baszczynski, et al., Plant Mol. Biol. 14: 633, 1990), Brazil Nut albumin (Pearson’ et al., Plant Mol. Biol. 18: 235-245, 1992), legumin (Ellis, et al. Plant Mol. Biol. 10: 203-214, 1988), Glutelin (rice) (Takaiwa, et al., Mol. Gen. Genet. 208: 15-22, 1986; Takaiwa, et al., FEBS Letts. 221: 43-47, 1987), Zein (Matzke et al., Plant Mol Biol, 143). 323-32 1990), napA (Stalberg, et al., Planta 199: 515-519, 1996), Wheat SPA (Albanietal, Plant Cell, 9: 171-184, 1997), sunflower oleosin (Cummins, et al., Plant Mol. Biol. 19: 873-876, 1992)], endosperm specific promoters [e.g., wheat LMW and HMW, glutenin-1 (Mol Gen Genet. 216:81-90, 1989; NAR 17:461-2), wheat a, b and g gliadins (EMBO3:1409-15, 1984), Barley ltrl promoter, barley B1, C, D hordein (Theor Appl Gen 98:1253-62, 1999; Plant J 4:343-55, 1993; Mol Gen Genet. 250:750-60, 1996), Barley DOF (Mena et al., The Plant Journal, 116(1): 53-62, 1998), Biz2 (EP99106056.7), Synthetic promoter (Vicente-Carbajosa et al., Plant J. 13: 629-640, 1998), rice prolamin NRP33, rice-globulin Glb-1 (Wu et al., Plant Cell Physiology 39(8) 885-889, 1998), rice alpha-globulin REB/OHP-1 (Nakase et al. Plant Mol. Biol. 33: 513-S22, 1997), rice ADP-glucose PP (Trans Res 6:157-68, 1997), maize ESR gene family (Plant J 12:235-46, 1997), sorgum gamma-kafirin (Plant Mol. Biol. 32:1029-35, 1996)], embryo specific promoters [e.g., rice OSH1 (Sato et al., Proc. Natl. Acad. Sci. USA, 93: 8117-8122), KNOX (Postma-Haarsma of al, Plant Mol. Biol. 39:257-71, 1999), rice oleosin (Wu et at, J. Biochem., 123:386, 1998)], and flower-specific promoters [e.g., AtPRP4, chalene synthase (chsA) (Van der Meer, et al., Plant Mol. Biol. 15, 95-109, 1990), LAT52 (Twell et al., Mol. Gen. Genet. 217:240-245; 1989), apetala-3].

Suitable abiotic stress-inducible promoters include, but not limited to, salt-inducible promoters such as RD29A (Yamaguchi-Shinozalei et al., Mol. Gen. Genet. 236:331-340, 1993); drought-inducible promoters such as maize rabl7 gene promoter (Pla et. al., Plant Mol. Biol. 21:259-266, 1993), maize rab28 gene promoter (Busk et. al., Plant J. 11:1285-1295, 1997) and maize Ivr2 gene promoter (Pelleschi et. al., Plant Mol. Biol. 39:373-380, 1999); heat-inducible promoters such as heat tomato hsp80-promoter from tomato (U.S. Pat. No. 5,187,267).

The term “Enzymatic activity” is meant to include demethylation, hydroxylation, epoxidation, N-oxidation, sulfooxidation, N—, S—, and O-dealkylations, desulfation, deamination, and reduction of azo, nitro, and N-oxide groups. The term “nucleic acid” refers to a deoxyribonucleotide or ribonucleotide polymer in either single- or double-stranded form, or sense or anti-sense, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof.

A “structural gene” is that portion of a gene comprising a DNA segment encoding a protein, polypeptide or a portion thereof, and excluding the 5′ sequence which drives the initiation of transcription. The structural gene may alternatively encode a nontranslatable product. The structural gene may be one which is normally found in the cell or one which is not normally found in the cell or cellular location wherein it is introduced, in which case it is termed a “heterologous gene”. A heterologous gene may be derived in whole or in part from any source known to the art, including a bacterial genome or episome, eukaryotic, nuclear or plasmid DNA, cDNA, viral DNA or chemically synthesized DNA. A structural gene may contain one or more modifications that could affect biological activity or its characteristics, the biological activity or the chemical structure of the expression product, the rate of expression or the manner of expression control. Such modifications include, but are not limited to, mutations, insertions, deletions and substitutions of one or more nucleotides. The structural gene may constitute an uninterrupted coding sequence or it may include one or more introns, bounded by the appropriate splice junctions. The structural gene may be translatable or non-translatable, including in an anti-sense orientation. The structural gene may be a composite of segments derived from a plurality of sources and from a plurality of gene sequences (naturally occurring or synthetic, where synthetic refers to DNA that is chemically synthesized).

“Derived from” is used to mean taken, obtained, received, traced, replicated or descended from a source (chemical and/or biological). A derivative may be produced by chemical or biological manipulation (including, but not limited to, substitution, addition, insertion, deletion, extraction, isolation, mutation and replication) of the original source.

“Chemically synthesized”, as related to a sequence of DNA, means that portions of the component nucleotides were assembled in vitro. Manual chemical synthesis of DNA may be accomplished using well established procedures (Caruthers, Methodology of DNA and RNA Sequencing, (1983), Weissman (ed.), Praeger Publishers, New York, Chapter 1); automated chemical synthesis can be performed using one of a number of commercially available machines.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, an “expression cassette” is a nucleic acid construct, generated recombinantly or synthetically, with a series of specified nucleic acid elements, which permit transcription of a particular nucleic acid in a target cell. The expression cassette can be incorporated into a plasmid, chromosome, mitochondrial DNA, plastid DNA, virus or nucleic acid fragment. Typically, the expression cassette portion of an expression vector includes, among other sequences, a nucleic acid to be transcribed and a promoter.

The terms “residue” or “amino acid residue” or “amino acid” are used interchangeably herein to refer to an amino acid that is incorporated into a protein, polypeptide or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)−0.61 (% form)—500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, N.Y. (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C. and a wash in 0.1×SSC, 0.1% SDS at 65° C.

As used herein, “transgenic plant” includes reference to a plant, which comprises within its genome a heterologous nucleic acid sequence. Generally, the heterologous nucleic acid sequence is stably integrated within the genome such that the nucleic acid sequence is passed on to successive generations. The heterologous nucleic acid sequence may be integrated into the genome alone or as part of a recombinant expression cassette. “Transgenic” is used herein to include any cell, cell line, callus, tissue, plant part or plant, the genotype of which has been altered by the presence of a heterologous nucleic acid sequence, including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

“Overexpression” refers to the level of expression in transgenic organisms that exceeds levels of expression in normal or untransformed organisms.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

“Preferred expression”, “Preferential transcription” or “preferred transcription” interchangeably refers to the expression of gene products that are preferably expressed at a higher level in one or a few plant tissues (spatial limitation) and/or to one or a few plant developmental stages (temporal limitation) while in other tissues/developmental stages there is a relatively low level of expression.

“Primary transformant” and “T0 generation” refer to transgenic plants that are of the same genetic generation as the tissue that was initially transformed (i.e., not having gone through meiosis and fertilization since transformation). “Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

A “selectable marker gene” refers to a gene whose expression in a plant cell gives the cell a selective advantage. The selective advantage possessed by the cells transformed with the selectable marker gene may be due to their ability to grow in presence of a negative selective agent, such as an antibiotic or a herbicide, compared to the ability to grow of non-transformed cells. The selective advantage possessed by the transformed cells may also be due to their enhanced capacity, relative to non-transformed cells, to utilize an added compound as a nutrient, growth factor or energy source. A selective advantage possessed by a transformed cell may also be due to the loss of a previously possessed gene in what is called “negative selection”. In this, a compound is added that is toxic only to cells that did not lose a specific gene (a negative selectable marker gene) present in the parent cell (typically a transgene).

The term “transformation” refers to the transfer of a nucleic acid fragment into the genome of a host cell, resulting in genetically stable inheritance. “Transiently transformed” refers to cells in which transgenes and foreign DNA have been introduced (for example, by such methods as Agrobacterium-mediated transformation or biolistic bombardment), but not selected for stable maintenance. “Stably transformed” refers to cells that have been selected and regenerated on a selection media following transformation.

“Transformed,” “transgenic,” and “recombinant” are used interchangeably and each refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

The term “translational enhancer sequence” refers to that DNA sequence portion of a gene between the promoter and coding sequence that is transcribed into RNA and is present in the fully processed mRNA upstream (5′) of the translation start codon. The translational enhancer sequence may affect processing of the primary transcript to mRNA, mRNA stability or translation efficiency. “Visible marker” refers to a gene whose expression does not confer an advantage to a transformed cell but can be made detectable or visible. Examples of visible markers include but are not limited to β-glucuronidase (GUS), luciferase (LUC) and green fluorescent protein (GFP).

“Wild-type” refers to the normal gene, virus, or organism found in nature without any mutation or modification.

As used herein, “plant material,” “plant part” or “plant tissue” means plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, tubers, rhizomes and the like.

As used herein “Protein extract” refers to partial or total protein extracted from a plant part. Plant protein extraction methods are well known in the art.

As used herein “plant sample” or “biological sample” refers to either intact or non-intact (e g milled seed or plant tissue, chopped plant tissue, lyophilized tissue) plant tissue. It may also be an extract comprising intact or non-intact seed or plant tissue. The biological sample or extract may be selected from the group consisting of corn flour, corn meal, corn syrup, corn oil, corn starch, and cereals manufactured in whole or in part to contain corn by-products.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, and 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG® programs (Accelrys, Inc., San Diego, Calif.).). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40 and 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (C₁₋ayerie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The phrase “abiotic stress” as used herein refers to any adverse effect on metabolism, growth, reproduction and/or viability of a plant by abiotic factors (i.e. water availability, heat, cold, and etc). Accordingly, abiotic stress can be induced by suboptimal environmental growth conditions such as, for example, salinity, water deprivation, water deficit, drought, flooding, freezing, low or high temperature (e.g., chilling or excessive heat), toxic chemical pollution, heavy metal toxicity, anaerobiosis, nutrient deficiency, nutrient excess, atmospheric pollution or UV irradiation.

The phrase “abiotic stress tolerance” as used herein refers to the ability of a plant to endure an abiotic stress without suffering a substantial alteration in metabolism, growth, productivity and/or viability.

As used herein “water deficit” means a period when water available to a plant is not replenished at the rate at which it is consumed by the plant. A long period of water deficit is colloquially called drought. Lack of rain or irrigation may not produce immediate water stress if there is an available reservoir of ground water to support the growth rate of plants. Plants grown in soil with ample groundwater can survive days without rain or irrigation without adverse effects on yield. Plants grown in dry soil are likely to suffer adverse effects with minimal periods of water deficit. Severe water deficit stress can cause wilt and plant death; moderate drought can reduce yield, stunt growth or retard development. Plants can recover from some periods of water deficit stress without significantly affecting yield. However, water deficit at the time of pollination can lower or reduce yield. Thus, a useful period in the life cycle of corn, for example, for observing response or tolerance to water deficit is the late vegetative stage of growth before tassel emergence or the transition to reproductive development. Tolerance to water deficit is determined by comparison to control plants. For instance, plants of this invention can produce a higher yield than control plants when exposed to water deficit. In the laboratory and in field trials drought can be simulated by giving plants of this invention and control plants less water than is given to sufficiently-watered control plants and measuring differences in traits. One aspect of the invention provides plants overexpressing the genes as disclosed herein which confers a higher tolerance to a water deficit.

As used herein, the phrase “water optimization” refers to any measure of a plant, its parts, or its structure that can be measured and/or quantified in order to assess an extent of or a rate of plant growth and development under different conditions of water availability. As such, a “water optimization trait” is any trait that can be shown to influence yield in a plant under different sets of growth conditions related to water availability. Exemplary measures of water optimization are grain yield at standard moisture percentage (YGSMN), grain moisture at harvest (GMSTP), grain weight per plot (GWTPN), and percent yield recovery (PYREC).

As used herein, the phrases “drought tolerance” and “drought tolerant” refer to a plant's ability to endure and/or thrive under conditions where water availability is suboptimal. In general, a plant is labeled as “drought tolerant” if it displays “enhanced drought tolerance.” As used herein, the phrase “enhanced drought tolerance” refers to a measurable improvement, enhancement, or increase in one or more water optimization phenotypes as compared to one or more control plants.

Water Use Efficiency (WUE) is a parameter frequently used to estimate the tradeoff between water consumption and CO₂ uptake/growth (Kramer, 1983, Water Relations of Plants, Academic Press p. 405). WUE has been defined and measured in multiple ways. One approach is to calculate the ratio of whole plant dry weight, to the weight of water consumed by the plant throughout its life (Chu et al., 1992, Oecologia 89:580). Another variation is to use a shorter time interval when biomass accumulation and water use are measured (Mian et al., 1998, Crop Sci. 38:390). Another approach is to utilize measurements from restricted parts of the plant, for example, measuring only aerial growth and water use (Nienhuis et al 1994 Amer J Bot 81:943). WUE also has been defined as the ratio of CO₂ uptake to water vapor loss from a leaf or portion of a leaf, often measured over a very short time period (e.g. seconds/minutes) (Kramer, 1983, p. 406). The ratio of ¹³C/¹²C fixed in plant tissue, and measured with an isotope ratio mass-spectrometer, also has been used to estimate WUE in plants using C-3 photosynthesis (Martin et al., 1999, Crop Sci. 1775). As used herein, the term “water use efficiency” refers to the amount of organic matter produced by a plant divided by the amount of water used by the plant in producing it, i.e. the dry weight of a plant in relation to the plant's water use. As used herein, the term “dry weight” refers to everything in the plant other than water, and includes, for example, carbohydrates, proteins, oils, and mineral nutrients. It is contemplated that the transgenic plants produced by the methods described herein will confer an increase in water use efficiency.

The phrase “biotic stress” as used herein refers to any adverse effect on metabolism, growth, reproduction and/or viability of a plant by biotic factors (i.e. insect pressure, disease and etc).

The phrase “biotic stress tolerance” as used herein refers to the ability of a plant to endure an biotic stress without suffering a substantial alteration in metabolism, growth, reproduction and/or viability.

As used herein the phrase “plant biomass” refers to the amount (measured in grams of air-dry or dry tissue) of a tissue produced from the plant in a growing season, which could also determine or affect the plant yield or the yield per growing area.

As used herein the phrase “plant vigor” refers to the amount (measured by weight) of tissue produced by the plant in a given time. Hence increased vigor could determine or affect the plant yield or the yield per growing time or growing area.

The term “early vigor” refers to active healthy well-balanced growth especially during early stages of plant growth, and may result from increased plant fitness due to, for example, the plants being better adapted to their environment (optimizing the use of energy resources and partitioning between shoot and root). Plants having early vigor also show increased seedling survival and a better establishment of the crop, which often results in highly uniform fields (e.g. crops growing in a uniform fashion, such as the crops reaching various stages of development at substantially the same time), and often higher yields. Therefore, early vigor may be determined by measuring various factors, such as thousand kernel weight, percentage germination, percentage emergence, seedling growth, seedling height, root length, root and shoot biomass and many more.

As used herein, “seedling vigor” refers to the plant characteristic whereby the plant emerges from soil faster, has an increased germination rate (i.e., germinates faster), has faster and larger seedling growth and/or germinates faster under cold conditions as compared to the wild type or control under similar conditions. Seedling vigor has often been defined to comprise the seed properties that determine “the potential for rapid, uniform emergence and development of normal seedlings under a wide range of field conditions”.

The life cycle of flowering plants in general can be divided into three growth phases: vegetative, inflorescence, and floral (late inflorescence phase). In the vegetative phase, the shoot apical meristem (SAM) generates leaves that later will ensure the resources necessary to produce fertile offspring. Upon receiving the appropriate environmental and developmental signals the plant switches to floral, or reproductive, growth and the SAM enters the inflorescence phase (I) and gives rise to an inflorescence with flower primordia. During this phase the fate of the SAM and the secondary shoots that arise in the axils of the leaves is determined by a set of meristem identity genes, some of which prevent and some of which promote the development of floral meristems. Once established, the plant enters the late inflorescence phase where the floral organs are produced. If the appropriate environmental and developmental signals are present the plant switches to floral, or reproductive, growth. If such signals are disrupted, the plant will not be able to enter reproductive growth, therefore maintaining vegetative growth.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells, which can be cultured into a whole plant.

Plants engineered for improved yield under various biotic and abiotic stresses is of special interest in the field of agriculture. For example, abiotic stress is a primary cause of crop loss worldwide, reducing average yields for most major crop plants by more than 50% (Wang et al., Planta (2003) 218: 1-14). Abiotic stresses may be caused by drought, floods, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

In some instances plant yield is relative to the amount of plant biomass a particular plant may produce. A larger plant with a greater leaf area can typically absorb more light, nutrients and carbon dioxide than a smaller plant and therefore will likely gain a greater weight during the same period (Fasoula & Tollenaar 2005 Maydica 50:39). Increased plant biomass may also be highly desirable in processes such as the conversion of biomass (e.g. corn, grasses, sorghum, cane) to fuels such as for example ethanol or butanol.

The ability to increase plant yield would have many applications in areas such as agriculture, the production of ornamental plants, arboriculture, horticulture, biofuel production, pharmaceuticals, enzyme industries which use plants as factories for these molecules and forestry. Increasing yield may also find use in the production of microbes or algae for use in bioreactors (for the biotechnological production of substances such as pharmaceuticals, antibodies, vaccines, fuel or for the bioconversion of organic waste) and other such areas.

Plant breeders are often interested in improving specific aspects of yield depending on the crop or plant in question, and the part of that plant or crop which is of relative economic value. For example, a plant breeder may look specifically for improvements in plant biomass (weight) of one or more parts of a plant, which may include aboveground (harvestable) parts and/or harvestable parts below ground. This is particularly relevant where the aboveground parts or below ground parts of a plant are for consumption. For many crops, particularly cereals, an improvement in seed yield is highly desirable. Increased seed yield may manifest itself in many ways with each individual aspect of seed yield being of varying importance to a plant breeder depending on the crop or plant in question and its end use.

It would be of great advantage to a plant breeder to be able to pick and choose the aspects of yield to be altered. It may also be highly desirable to be able to pick a gene suitable for altering a particular aspect of yield (e.g. seed yield, biomass weight, water use efficiency, yield under stress conditions). For example an increase in the fill rate, combined with increased thousand kernel weight would be highly desirable for a crop such as corn. For rice and wheat a combination of increased fill rate, harvest index and increased thousand kernel weight would be highly desirable.

It has now been discovered that the expression of several forms of trehalose-6-phosphate phosphatase (T6PP) in plants confers a significant increase in yield as well as confer resistance to various types of stress (i.e. abiotic stress). During the course of analyzing various T6PP variants transgenically expressed in plants it has been found that the transgenic plants showing the highest yield in seed also comprised T6PPs with modifications to amino acid residues associated with substrate binding. Not to be limited by theory, these proteins may have decreased activity as compared to a T6PP not containing these modifications. Further, not to be limited by theory, it appears that expressing a T6PP in plant with decreased activity results in a beneficial phenotype having significant increased yield in both stress (e.g. drought) and non-stressed field conditions. T6PP is an enzyme involved in the trehalose biosynthesis pathway. Trehalose, a non-reducing disaccharide consisting of two glucose molecules linked via alpha-1,1 bonds. The sugar trehalose can be found in many various organisms across multiple kingdoms (e.g. plants, bacteria, insects, etc). Trehalose has been shown to be involved in carbohydrate storage function and has been further associated to play a role in stress tolerance in bacteria, fungi and insects. In plants, trehalose was initially thought to be confined to extremophiles such as the resurrection plant Selaginella lepidophylla, however it is now widely accepted that trehalose metabolism is ubiquitous in the plant kingdom.

Trehalose is synthesized from UDP-glucose and Glucose-6-phosphate in two enzymatic reactions. First UDP-glucose and Glucose-6-phosphate are converted to UDP (uridine diphosphate) and alpha, alpha-trehalose 6-phosphate (T6P) by the enzyme T6PS (trehalose phosphate synthase). In a second step, which is catalyzed by the enzyme T6PP (trehalose phosphate phosphatase), T6P is de-phosphorylated to produce trehalose and orthophosphate.

In yeast, the two enzymatic activities (T6PS and T6PP activity) reside in a large protein complex, containing the active subunits, T6PS 1 and T6PS2, and the regulatory subunits, with T6PS1 having T6PS activity and T6PS2 having T6PP activity. In E. coli, the two enzymatic activities are found in separate protein complexes. In plants, the protein complex has not been characterized to date.

In Arabidopsis thaliana, trehalose biosynthetic enzymes have been classified into three classes:

Class I: containing four genes, AtT6PS1 to AtT6PS4 having high similarity to ScT6PS1;

Class II: having seven members, AtT6PS5 to AtT6PS11, with high sequence similarity to ScT6PS2; and

Class III:, containing 10 members, AtT6PPA to AtT6PPJ, encoding proteins with similarity to E. coli T6PS2 and the C-terminus of ScT6PS2 proteins.

Genes encoding proteins within these classes are also present in other plant species.

Within Class I and Class II, enzymatic activity has only been unambiguously determined for AtT6PS1, which displays T6PS activity (Blazquez et al. Plant J. March 1998; 13(5):685-9.). Surprisingly, no T6PP activity has been reported to date for any of the other Class II T6PS proteins. In contrast, T6PP activity was previously described for AtT6PPA and AtT6PPB, two of the members of Class III (Vogel et al. Plant J. March 1998; 13(5):673-83). Plant Class III T6PPs contain two phosphatase consensus sequence motifs found in all T6PP enzymes described to date (Thaller et al. Protein Sci. July 1998; 7(7):1647-52).

The genetic manipulation of trehalose biosynthesis genes has been reported to lead to improved stress tolerance in plants, as well as causing striking developmental alterations. Overexpression of E. coli OtsA and OtsB genes (equivalents to T6PP and T6PS) in transgenic tobacco and potato plants was reported to cause developmental aberrations in roots and leaves as well as stunted plant growth. Fewer seeds were produced in the OtsA transgenic tobacco plants and the OtsB transgenic potato plants did not produced tubers (Goddijn et al. Plant Physiol. January 1997; 113(1):181-90). Similar results have been described by others (Holmstrom et al. Nature, 379, 683-684; Romero et al. Planta, 201, 293-297; Pilont-Smits et al. 1998; J Plant Physiol. 152:525-532; Schluepmann et al. Proc Natl. Acad. Sci. USA. 2003; 100(11):6849-54). Mutants defective in T6PS and T6PP genes have also reportedly shown developmental defects. T6PS 1 knock out mutants in Arabidopsis showed impaired embryo development (Eastmond et al. Plant J. January 2002; 29(2):225-35). McSteen et. al. (Plant Cell 2006; 18; 518-522) mentions the isolation and characterization of a maize geneRAMOSA3 (RA3) reported to be responsible for meristem development and inflorescence development including branching. It is suggested that the gene, gene product, and regulatory regions may be used to manipulate branching, meristem growth, inflorescence development and arrangement. Negative phenotypes associated with the expression of a transgene can have detrimental effects to a plant's relative yield. For example, without seed set, seed filling, fertility of a plant etc. there would be no increase in seed yield. Patent application U.S. 2007/0006344 is a first account to this application's knowledge of a method which describes the expression of a T6PP in a plant to confer an increase in plant yield without any negative phenotypes and/or detrimental effects to the plant biological function. U.S. Patent Application 2007/0006344 describes the use of a trehalose-6-phosphate phosphatase operably linked to a OsMADS promoter that targets the preferential expression of the T6PP to maternal reproductive tissue of a plant which resulted in a significant yield increase in maize under stress and non-stress conditions. The following invention generally involves the identification of T6PP having modifications that confer improved yield and field efficacy in crop plants and further describes methods one may use to increase yield in a plant by utilizing modified T6PPs in a plant.

As used herein the term “reduced activity” refers to any decrease in T6PP activity.

T6PP proteins (at least in their native form) typically have trehalose-6-phosphate phosphatase activity. Polypeptides with trehalose-6-phosphate phosphatase activity belong to the enzymatic class of EC:3.1.3.12, according to the classification of the Enzyme Commission of the Nomenclature Committee of the International Union of Biochemistry and Molecular Biology (NC-IUBMB). Enzymes in class EC:3.1.3.12 catalyze the reaction: trehalose-6-phosphate+H₂O=trehalose+phosphate. It is contemplated that any T6PP or protein having T6PP activity can be modified for decreased activity via methods such as point mutation(s), irradiation, etc to confer the positive effects as described herein when expressed transgenically in a plant.

The activity of a trehalose-6-phosphate phosphatase protein may be measured by determining the levels of the substrate processed and the levels of product accumulated in an in vitro reaction, that is, by determining the level of trehalose-6-phosphate consumption and/or trehalose accumulation from the reaction. Enzymatic methods to measure trehalose can be based on hydrolyzing trehalose to glucose, such as those described by Van Dijck et al. Biochem J. Aug. 2002 15; 366(Pt 1):63-71 and Zentella et al. Plant Physiol. April 1999; 119(4):1473-82.

Trehalose-6-phosphate levels may also be measured by HPLC (High Performance Liquid Chromatography) methods as described by Avonce et al. Plant Physiol. November 2004; 136(3):3649-59; Schluepmann et al. 2003. Alternative methods based on determining the release of inorganic phosphate from trehalose-6-phosphate have also been described Klutts et al. J Biol. Chem. January 2003 24; 278(4):2093-100. An alternative method to determine trehalose-6-phosphate levels using liquid chromatography coupled to MS-Q3 (triple quadrupole MS) has been described by Lunn et al. Biochem J. July 2006 1; 397(1):139-48.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

The nucleic acid may be introduced directly into a plant cell or into the plant itself (including introduction into a tissue, organ or any other part of a plant). According to a preferred feature of the present invention, the nucleic acid is preferably introduced into a plant by transformation.

The transfer of foreign genes into the genome of a plant, other than by breeding, is called transformation. Any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen. Genet. 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

The genetically modified plant cells can be regenerated via all methods with which the skilled worker is familiar Suitable methods can be found in the above-mentioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organization. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a T6PP protein as defined hereinabove. Preferred host cells according to the invention are plant cells.

Host plants for the nucleic acids or the vector used in the method according to the invention, the expression cassette or construct or vector are, in principle, advantageous in all plants, which are capable of synthesizing the polypeptides used in the inventive method.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

The invention also extends to harvestable parts of a plant such as, but not limited to seeds, leaves, ears, flowers, stems, and other biological samples. The invention furthermore relates to products derived, preferably directly derived, from a harvestable part of such a plant, such as dry pellets or powders, oil, fat and fatty acids, starch, proteins, or an extract derived from corn event MZDT09Y. The biological sample or extract is selected from the group consisting of corn flour, corn meal, corn syrup, corn oil, corn starch, and cereals manufactured in whole or in part to contain corn-by-products.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of suitable control plants.

The terms “increase”, “improving” or “improve” are interchangeable and shall mean in the sense of the application at least a 5%, 6%, 7%, 8%, 9% or 10%, preferably at least 15% or 20%, more preferably 25%, 30%, 35% or 40% more yield and/or growth in comparison to the wild type plant as defined herein.

Increased seed yield may manifest itself as one or more of the following: a) an increase in seed biomass (total seed weight) which may be on an individual seed basis and/or per plant and/or per hectare or acre; b) increased number of flowers per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; and f) increased thousand kernel weight (TKW), which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

Since the corn event MZDT09Y plants according to the present invention have increased yield, it is likely that these plants exhibit an increased growth rate (during at least part of their life cycle), relative to the growth rate of control plants at a corresponding stage in their life cycle. The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigor, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigor. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soy bean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

Performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a T6PP protein as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects. Another abiotic stress may result from a nutrient deficiency, such as a shortage of nitrogen, phosphorus and potassium.

The methods of the present invention may be performed under non-stress conditions or under conditions of mild drought to confer plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinization are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signaling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under drought conditions increased yield relative to suitable control plants grown under comparable conditions. Therefore, according to the present invention, there is provided a method for enhancing yield-related traits in plants grown under non-stress conditions or under drought conditions, which method comprises increasing expression in a plant of a nucleic acid encoding a modified T6PP polypeptide having decreased substrate binding and/or activity.

In one embodiment of the invention, the enhanced yield-related trait is manifested as an increase in one or more of the following: total number of seeds per plant, number of filled seeds per plant and seed weight per plant. Preferably, these increases are found in plants grown under non-stress conditions.

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

A variety of nucleic acid amplification-based methods for genetic and physical mapping may be carried out using nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### EXAMPLES

### Example 1

### Identification and Cloning of the Rice T6PP cDNA Sequence into a Binary Vector

The first vascular plant trehalose-6-phosphate phosphatase genes were cloned from Arabidopsis thaliana by complementation of a yeast T6PS2 deletion mutant (Vogel et al. 1998). The genes designated AtT6PPA and AtT6PPB (GenBank accessions AF007778 and AF007779) were shown at that time to have trehalose-6-phosphate phosphatase activity. The AtT6PPA and AtTTPB protein sequences were used in TBLASTN queries of maize and rice sequence databases. Sequence alignments organized the hits into individual genes. Three maize and three rice T6PP homologs were identified. The rice T6PP (OsT6PP) cDNA sequence as indicated by SEQ ID NO: 29 was amplified using high-fidelity PCR. The 50 μL reaction mixture consisted of 1 μL rice cDNA library (prepared from callus mRNA in Stratagene's Lambda Unizap Vector, primary library size >1×10⁶ pfu, amplified library titer >1×10¹² pfu/mL), 200 μM dNTPs, 1 μL 20 μM of oligonucleotide primer T6PP-EC-5 (5′-catggaccatggatttgagcaatagctcac-3′) and 1 μL 20 μM of oligonucleotide primer T6PP-EC-3 (5′-atcgcagagctcacactgagtgcttcttcc-3′), 5 μL 10×Cloned PFU buffer and 2.5 Units of Pfuturbo DNA polymerase. The thermocycling program was 95° C. for 2 minutes followed by 40 cycles of (94° C. for 15 seconds, 50° C. for 1 minute, 72° C. for 1 minute) followed by 72° C. for 10 minutes. The rice T6PP product was cloned with the Zero Blunt TOPO PCR cloning kit. The pCR-Blunt-II-TOPO-OsT6PP is identified by digesting 5 μL pCR-Blunt-II-TOPO-OsT6PP miniprep DNA with EcoRI in a 20 μL reaction containing 2 μg BSA and 2 μL 10×EcoRI restriction endonuclease buffer. The reaction is incubated at 37° C. for 2 hours and the pCR-Blunt-II-TOPO-OsT6PP (EcoRI) products are resolved on 1% TAE agarose. The pCR-Blunt-II-TOPO-OsT6PP clone is then sequenced. The OsT6PP cDNA is flanked by NcoI/SacI restriction endonuclease sites. The OsT6PP was then further cloned into a binary vector as described in Example 8 of U.S. Patent Application Publication 2007/0006344 (therein referred to OsT6PP-3 and indicated by nucleotide SEQ ID NO: 531 and protein SEQ ID NO: 532)

### Example 2

### Initial Evaluation of Rice T6PP Maize Events in the Greenhouse

Rice T6PP maize events comprising SEQ ID NO: 1 operably linked to a promoter having preferential expression in maternal reproductive tissue (i.e. OsMADS promoter) were generated and further evaluated in both the greenhouse and field as described in Examples 8-13 in U.S. Patent Application Publication 2007/0006344. Initial greenhouse and field evaluation of the maize events indicated some events having a yield increase in both non-drought and drought conditions (See U.S. Patent Application Publication 2007/0006344 herein incorporated by reference).

### Example 3

### Evaluation and Identification High Yielding T6PP Maize Events

The maize events shown to confer a yield increase in the trials described in Example 2 and more specifically in U.S. Patent Application Publication 2007/0006344, were further characterized for yield and field efficacy. These events contained either binary construct 15777 (SEQ ID NO: 30) or 15769 (SEQ ID NO: 31) as is described in U.S. Patent Application Publication 2007/0006344. Essentially binary construct 15769 comprises an expression cassette having a OsT6PP (indicated in SEQ ID NO: 29 of the current application) operably linked to a OsMADS6 promoter. Binary construct 15777 contains the same expression cassette (OsMADS6 promoter and OsT6PP coding sequence) with the addition of transcriptional enhancers upstream of the OsMADS6 promoter. The details and specifics of both these constructs may again be found in the U.S. Patent Application Publication 2007/0006344. Overall there were 645 T0 maize events generated from 15769 and 587 maize events were generated comprising the 15777 binary construct. Following the course of generation of transgenic events during plant transformation, selection of events having successfully integrated into the genomic DNA, as well as growth in greenhouse and field conditions relatively a small number of events were carried forward for field trials based on the selection criteria as well as plant event survival and phenotype criteria. The relatively high level of attrition led to only 17 events showing field efficacy. Events derived from maize plants comprising the 15777 binary construct proved to be most efficacious in the field testing. Two events, MZDT09Y and MZDT08H, were selected based upon viability and performance in managed stress environments (yield preservation under drought at flowering) and in agronomic trials which measured yield. Overall, best performing events comprising construct 15777 demonstrated a significant bushel per acre yield advantage over control check samples.

### Example 4

### Corn Events MZDT09Y and MZDT08H Sequence Analysis

Corn event MZDT09Y and corn event MZDT08H were further analyzed by sequencing of the T6PP CDS. PCR was used to amplify the integrated OsT6PP coding sequence using primers that anneal to the 5′ and 3′ region of the coding sequence. The respective PCR amplicons resulted in the approximate 1.1 Kb band size as would be expected from the coding sequence of the OsT6PP as depicted in SEQ ID NO: 29 which was the sequence that was comprised in the relative expression cassette. The amplicons were further sequenced as is well established in the art. Sequencing data indicated that both events contained modifications. MZDT09Y contained a single point mutation at nucleotide 730 respective to SEQ ID NO: 29 (T*CATTA where * indicates point of mutation). This single mutation led to an amino acid mutation changing a His residue to an Asp residue. MZDT08H was found to contain two modifications at nucleotides 305 (TG*CTTCC where * indicates point of mutation) and 388 (CGCC*ATT where * indicates point of mutation) respective to SEQ ID NO: 29. Interestingly, these mutations are located in highly conserved domains of the T6PP protein.

Primer pairs used to sequence the heterologous insert are as follows: 09Y-1S-1 (SEQ ID NO: 26) was amplified using forward primer 09Y-RBFS-F1 (SEQ ID NO: 16) and reverse primer 09Y-1S-R1 (SEQ ID NO: 18); 09Y-1S-2 (SEQ ID NO: 27) was amplified using forward primer 09Y-1S-F2 (SEQ ID NO: 19) and reverse primer 09Y-1S-R2 (SEQ ID NO: 20); 09Y-1S-3 (SEQ ID NO: 28) was amplified using forward primer 09Y-1S-F6 (SEQ ID NO: 21) and reverse primer 09Y-1S-R6 (SEQ ID NO: 22).

The modified T6PP enzyme (SEQ ID NO: 11) of corn event MZDT09Y has approximately 10% activity relative to the unmodified T6PP enzyme, and yet corn event MZDT09Y is not affected by yield drag. However, the modified T6PP enzyme of MZDT08H does impart a slight yield drag on the corn plant. For this surprising and unexpected result, corn event MZDT09Y was selected for progression.

### Example 5

### Methods of Detecting Corn Event MZDT09Y by PCR

Corn event MZDT09Y can be detected by assaying for a nucleotide sequence comprising SEQ ID NO: 1, SEQ ID NO: 2, SEQ ID NO: 3, or SEQ ID NO: 4. Polymerase chain reaction (PCR) is a standard method of amplifying DNA practiced by those of skill in the art, provided that the sequence of the template DNA is known. SEQ ID NOs: 1-4 are completely unique to MZDT09Y and are disclosed herein for the first time. Primer pairs different from those primers explicitly disclosed herein may be developed by one of ordinary skill in the art and still achieve the same function, that is, to identify corn event MZDT09Y.

Primers SEQ ID NO: 12 (forward primer MZDT09Y-RB1) and SEQ ID NO: 13 (reverse primer ESPCR0001) function together in a PCR reaction to produce a right border amplicon comprising SEQ ID NO: 23 and its complementary sequence, which is indicative of the presence of MZDT09Y template DNA. This amplicon also comprises SEQ ID NO: 1 and SEQ ID NO: 3.

Primers SEQ ID NO: 32 (forward primer) and SEQ ID NO: 36 (reverse primer) function together in a PCR reaction to produce a right border amplicon comprising SEQ ID NO: 37 and its complementary sequence, which is indicative of the presence of MZDT09Y template DNA. This amplicon also comprises SEQ ID NO: 1.

Primers SEQ ID NO: 14 (forward primer 09Y-LBFS-F1) and SEQ ID NO: 15 (reverse primer 09Y-LBFS-R4) function together in a PCR reaction to produce a left border amplicon comprising SEQ ID NO: 24 and its complementary sequence, which is indicative of the presence of MZDT09Y template DNA. This amplicon also comprises SEQ ID NO: 2 and SEQ ID NO: 4.

In addition to conventional, gel-based PCR, TaqMan® PCR (Invitrogen™) which uses fluorescence to enable detection of amplification in real-time, may also be used to detect the presence of corn event MZDT09Y. Forward primer P23198 (SEQ ID NO: 32), reverse primer P23352 (SEQ ID NO: 33), and probe P23200 (SEQ ID NO: 34, labeled with FAM on its 5′-terminus, and with BHQ1 on its 3′-terminus) function together in a TaqMan PCR reaction in the presence of MZT09Y template DNA to produce an amplicon (SEQ ID NO: 35), and thereby fluorescence, diagnostic of corn event MZDT09Y.

### Example 6

### Methods of Detecting Corn Event MZDT09Y by Hybridization

Corn event MZDT09Y can be detected by assaying using hybridization techniques for a nucleotide sequence comprising SEQ ID NO: 1, SEQ ID NO: 2, SEQ ID NO: 3, or SEQ ID NO: 4. Methods of hybridization, for example a Southern blot, can detect DNA, provided that the sequence of the template DNA is known. SEQ ID NOs: 1-4 are completely unique to MZDT09Y and are disclosed herein for the first time.

By way of example and not limitation, DNA samples are cut with restriction enzymes and run overnight on an agarose gel in 1×TBE buffer at about 32 volts. Gels are photographed, washed, and blotted onto nylon membrane with 10×SSC as the transfer solution. They are linked to the membrane with UV light and pre-hybridized with calf thymus DNA at 65° C. Probes comprising SEQ ID NO: 1 or SEQ ID NO: 2 are labeled with radioactive Phosphorus 32. Probes are added and hybridized at 65° C., 3 hrs to overnight. Blots are washed several times and exposed in a phosphorimager cassette. Images are developed and scored.

### Deposit

Applicants have made a deposit of corn seed of event MZDT09Y disclosed above on Jun. 28, 2012, in accordance with the Budapest Treaty at the American Type Culture Collection (ATCC), 10801 University Boulevard, Manassas, Va., 20110, USA under ATCC Accession No. PTA-13025. The deposit will be maintained in the depository for a period of 30 years, or 5 years after the last request, or the effective life of the patent, whichever is longer, and will be replaced as necessary during that period. Applicants impose no restrictions on the availability of the deposited material from the ATCC; however, Applicants have no authority to waive any restrictions imposed by law on the transfer of biological material or its transportation in commerce. Applicants do not waive any infringement of their rights granted under this patent or under the Plant Variety Protection Act (7 USC 2321 et seq.).

All publications and published patent documents cited in this specification are incorporated herein by reference to the same extent as if each individual publication or patent document was specifically and individually indicated to be incorporated by reference.

Although the foregoing invention has been described in some detail by way of illustration and example for purposes of clarity and understanding, it will be clear to those of skill in the art that certain changes and modifications may be practiced within the scope of the appended claims.

What is claimed is:

1. A pair of polynucleotide primers comprising a first polynucleotide primer and a second polynucleotide primer which function together in the presence of an event MZDT09Y DNA template in a sample to produce an amplicon diagnostic for event MZDT09Y, wherein
a. the first polynucleotide primer comprises at least 10 contiguous nucleotides of SEQ ID NO: 36 or the complement thereof; and
b. the second polynucleotide primer comprises a nucleotide sequence selected from the group comprising SEQ ID NO: 1, SEQ ID NO: 32, or the complements thereof.

2. A method of detecting the presence of a nucleic acid molecule that is unique to event MZDT09Y in a sample comprising corn nucleic acids, the method comprising:
a. contacting the sample with the primer pair of claim 1, which when used in a nucleic acid amplification reaction with genomic DNA from event MZDT09Y produce an amplicon that is diagnostic for event MZDT09Y;
b. performing a nucleic acid amplification reaction, thereby producing the amplicon; and
c. detecting the amplicon.

3. A kit for detecting nucleic acids that are unique to event MZDT09Y comprising at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to event MZDT09Y in the sample, wherein the nucleic acid molecule is selected from the group consisting of SEQ ID NO: 1, SEQ ID NO: 32, SEQ ID NO: 36, and the complements thereof.
